SEQUENCE LISTING (14 sequences)

SEQ ID NO: 1 - DNA, 923 bp, Mus musculus
agactatacaggaattgggactttctgggagcatctctctcagcgctggtagggtaccctaaggatgaggagagactaaatgggggttttcctgctgagccaggccatgctgactgcaatgttgctgagttgtgttctgctgttggcactgcctcccacactgggggtccagatgggcgtggctccactgaagggcatcagaaggcctgaccaggctctgttcccagagttcccaggtgagtatggtcaggttggggatatgtggggcaacgaccattgctggccacagacctgcccgcccaggcttagacctccttccccaatcccaatcccaacctagggaggtgggtacttggtgcatggtgggtgtggccctcacatcttcttgccccaggtctaagtctgaatggcctcaagaagacaactgcagaccgagcagaagaagttctgctgcagaaggcagaagctttggcggaggtaactcattagggaaagggataaagtagaaggtagggcgcatcagataccatcatctctccccacttccggattacccaacctgggcagaactgcagcccctccctgacctcagtccactgccaccctactggggtcggggtttgagagtttcctgaaccttattcccctacgaatgcaggtgctagatccacagaaccgcgagtctcgttctccgcgtcgctgtgtaaggctgcacgagtcctgcttgggacagcaggtaccttgctgcgacccgtgcgctacgtgctactgccgcttcttcaatgccttttgctactgccgcaagctgggtaccgccacgaacctctgtagtcgcacctagccaatggatgttgtttgggaaaggcaggggatgagaataaaggatcgggacggtttaaccttaaagctgtggttatttcttt

SEQ ID NO: 2 - protein, 131 aa, Mus musculus
MLTAMLLSCVLLLALPPTLGVQMGVAPLKGIRRPDQALFPEFPGLSLNGLKKTTADRAEEVLLQKAEALAEVLDPQNRESRSPRRCVRLHESCLGQQVPCCDPCATCYCRFFNAFCYCRKLGTATNLCSRT

SEQ ID NO: 3 - DNA, 486 bp, Homo sapien
gccatgctgaccgcagcggtgctgagctgtgccctgctgctggcactgcctgccacgcgaggagcccagatgggcttggcccccatggagggcatcagaaggcctgaccaggccctgctcccagagctcccaggcctgggcctgcgggccccactgaagaagacaactgcagaacaggcagaagaggatctgttgcaggaggctcaggccttggcagaggtactagacctgcaggaccgcgagccccgctcctcacgtcgctgcgtaaggctgcatgagtcctgcctgggacagcaggtgccttgctgtgacccatgtgccacgtgctactgccgcttcttcaatgccttctgctactgccgcaagctgggtactgccatgaatccctgcagccgcacctagctggccaacgtcagggtcggggctagggtaggggcaaggaaactcgaataaaggatgggaccaacaaaaaaaaaaaaaaaaaaa

SEQ ID NO: 4 - protein, 132 aa, Homo sapien
MLTAAVLSCALLLALPATRGAQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT

SEQ ID NO: 5 - DNA, 483 bp, Mus musculus
atgctaggtaacaagcgaatggggctgtgtggactgaccctcgctctatctctgctcgtgtgtttgggcattctggctgaggggtacccctccaagccggacaatccgggcgaggacgcgccagcagaggacatggccagatactactccgctctgcgacactacatcaatctcatcaccagacagagatatggcaagagatccagccctgagacactgatttcagacctcttaatgaaggaaagcacagaaaacgcccccagaacaaggcttgaagacccttccatgtggtgatgggaaatgaaacttgttctcccgacttttccaagtttccaccctcatctcatctcatccatcccctgaaaccagtctgcctgtcccaccaatgcatgccaccactaggctggactccgccccatttcccttgttgttgttgtatatatgtgtgtttaaataaagtaccatgcattcaaaaaaaaaaaa

SEQ ID NO: 6 - protein, 97 aa, Mus musculus
MLGNKRMGLCGLTLALSLLVCLGILAEGYPSKPDNPGEDAPAEDMARYYSALRHYINLITRQRYGKRSSPETLISDLLMKESTENAPRTRLEDPSMW

SEQ ID NO: 7 - DNA, 404 bp, Homo sapien
atgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattc

SEQ ID NO: 8 - protein, 97 aa, Homo sapien
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 9 - DNA, 402 bp, Artificial Sequence
  Probe
catgctgaccgcaatgttgctgagttgtgttctgctgttggcactgcctcccacactgggggtccagatgggcgtggctccactgaagggcatcagaaggcctgaccaggctctgttcccagagttcccaggtctaagtctgaatggcctcaagaagacaactgcagaccgagcagaagaagttctgctgcagaaggcagaagctttggcggaggtgctagatccacagaaccgcgagtctcgttctccgcgtcgctgtgtaaggctgcacgagtcctgcttgggacagcaggtaccttgctgcgacccgtgcgctacgtgctactgccgcttcttcaatgccttttgctactgccgcaagctgggtacggccacgaacctctgcagccgcacctagccaat

SEQ ID NO: 10 - DNA, 45 bp, Artificial Sequence
  Antisense Oligonucleotides
tgcagcagaacttcttctgctcggtctgcagttgtcttcttgagg

SEQ ID NO: 11 - DNA, 45 bp, Artificial Sequence
  Antisense Oligonucleotides
agcttgcggcagtagcaaaaggcattgaagaagcggcagtagcac

SEQ ID NO: 12 - DNA, 23 bp, Artificial Sequence
  Primer
aaatcagaaggccacaccccggt

SEQ ID NO: 13 - DNA, 25 bp, Artificial Sequence
  Primer
aaatcgaccgcgtggtggtgctaat

SEQ ID NO: 14 - DNA, 25 bp, Artificial Sequence
  Primer
taaagcgcatgctccagactgcctt

(19) United States
(12) Patent Application Publication
(10) Pub. No.: US 2005/0257279 A1
(43) Pub. Date: Nov. 17, 2005

(54) Agouti-related protein deficient cells, non-human transgenic animals and methods of selecting compounds which regulate energy metabolism

(76) Inventors: Su Qian, Princeton, NJ (US); Leonardus H.T. Van der Ploeg, Scotch Plains, NJ (US); Howard Y. Chen, Westfield, NJ (US); Drew T. Weingarth, East Brunswick, NJ (US); Myrna E. Trumbauer, Yardley, PA (US); Joseph M. Metzger, Fanwood, NJ (US)

Correspondence Address: MERCK AND CO., INC, P O BOX 2000, RAHWAY, NJ, 07065-0907, US

(21) Appl. No.: 10/518,955

(22) PCT Filed: Jun. 27, 2003
(86) PCT No.: PCT/US03/20245

§ 371 Date: Dec. 17, 2004

Related U.S. Application Data
(60) Provisional application No. 60/393,391, filed on Jul. 3, 2002.

(51) Int. Cl. A01K 67/027; C12N 5/06

(52) U.S. Cl. 800/18; 435/354

(57) ABSTRACT

Cells and non-human transgenic animals have been engineered to be deficient in the gene encoding agouti-related protein (AgRP). AgRP deficient transgenic animals have a reduced day time respiratory quotient (RQ), indicating that AgRP is involved in the regulation of energy metabolism, resulting in the reduced usage of fat as an energy source. These AgRP deficient transgenic animals can be used to select for and test potential modulators of AgRP. This data allows for methods of screening for AgRP modulators which regulate energy metabolism and caloric utilization. The disclosure also relates to a NPY/AgRP double knockout mouse which can be used to select for and test potential modulators (e.g., agonists or antagonists) of AgRP and/or NPY.

### CROSS-REFERENCE TO RELATED APPLICATIONS

The present application claims the benefit U.S. Provisional Application No. 60/393,391, filed Jul. 3, 2002, hereby incorporated by reference herein.

### STATEMENT REGARDING FEDERALLY-SPONSORED R&D

Not Applicable

### REFERENCE TO MICROFICHE APPENDIX

Not applicable.

### FIELD OF THE INVENTION

The present invention relates to cells and non-human transgenic animals that have been engineered to be deficient in the gene encoding agouti-related protein (AgRP). It is shown herein that AgRP deficient transgenic animals exhibit a reduced daytime respiratory quotient (RQ). Respiratory quotient is a measure which, in general, can be used to indicate whether carbohydrate or fat is being used as an energy source. The reduced daytime RQ identified in AgRP^{-/-} mice indicates that these mice preferentially oxidize fat during the day. Relative utilization of fat versus carbohydrate can be estimated from RQ measurements using the simplified model of carbohydrate and fat oxidation proposed by Elia and Livesey (1992) World Rev Nutr Diet 70:68-131 under the assumption of steady-state protein oxidation as proposed by Flatt et al., (1991) J. Nutr Biochem 2:193-202. Employing this model, it was determined that AgRP^{-/-} mice (which are disclosed herein) derive approximately 20% more of their daytime energy from fat when compared to wild-type control mice; a finding implicating AgRP in the regulation of caloric utilization, and particularly in the reduced usage of fat as an energy source. This is consistent with the bulk of data indicating that AgRP increases fat storage; see, e.g., Chen et al., (2000) Regul Pept 92(1-3):113-9.

AgRP deficient transgenic animals can be used to select for and test potential modulators (e.g., agonists or antagonists) of AgRP. AgRP deficient transgenic animals can also be used to select for dual modulators, for example, modulators of both AgRP and NPY when the AgRP transgenic animal is deficient in neuropeptide Y (NPY) as well. The neuropeptide Y (NPY)/AgRP double knockout mouse disclosed herein is useful in that regard. The deficient transgenic animals can also be used to select for and test potential modulators (e.g., agonists) of melanocortin (MC) receptors MC-4R and MC-3R, as lack of the endogenous antagonist (AgRP) would predict similar efficacy at lower agonist concentrations. The NPY/AgRP double knockout mouse disclosed herein can also be used to select for and test potential modulators (e.g., agonists) of ghrelin-R, as NPY and AgRP are mediators of orexingenic actions of peripheral ghrelin. To this end, the present invention relates further to methods of screening for AgRP, NPY, MC-4R and/or MC-3R, ghrelin-R modulators which effect energy metabolism and associated methods of treating various disorders or diseases responsive to the action of one or more of the neuropeptides or melanocortin receptors, including but not limited to obesity (by reducing appetite, increasing metabolic rate, reducing fat intake or reducing carbohydrate craving), diabetes mellitus (by enhancing glucose tolerance, decreasing insulin resistance), hypertension, hyperlipidemia, osteoarthritis, cancer, gall bladder disease, male and female sexual dysfunction (including impotence, loss of libido and erectile dysfunction), modulation of cytokine release, skin tanning, acne and other skin disorders.

### BACKGROUND OF THE INVENTION

Agouti-related protein (AgRP; Shutter et al., (1997) Genes & Dev. 11:593-602), a 132-amino acid neuropeptide abundantly expressed in the arcuate nucleus of the hypothalamus, potently stimulates feeding and body weight gain in rodents. In fact, overexpression of AgRP recapitulates many of the features present in the obese yellow (Ay/a) and MC-4R-deficient mice including obesity, increased body length, hyperinsulinaemia, late-onset hyperglycaemia, pancreatic-islet hyperplasia and lack of elevated corticosterone; see (Graham et al., (1997) Nature Genetics 17:273-274; Ollmann et al., (1997) Science 278:135-138). AgRP is believed to function via antagonizing α-melanocyte-stimulating hormone (α-MSH) signaling at the central nervous system (CNS) melanocortin-3 and -4 receptors (MC-3R, MC-4R). Antagonism of this interaction counteracts the effects normally attributed to α-MSH signaling—namely, reduced food intake and increased energy expenditure.

AgRP is exclusively expressed in a subset of hypothalamic arcuate nucleus neurons. The only peripheral tissue known to express AgRP is the adrenal gland. AgRP is detected at a low level in the plasma of rats and humans. Plasma AgRP levels in these two species are affected by meal consumption and it has been postulated that plasma AgRP levels may reflect satiety.

AgRP is 25% homologous to the protein agouti, particularly in the cysteine-rich C-terminal domain. Agouti is a 131 amino acid paracrine hormone normally expressed in the skin. Therein, agouti controls hair color through antagonism at MC-1R, a G protein-coupled receptor of the hair follicle; (Bultman et al., (1992) Cell 71:1195-1204). Ectopic expression of agouti in the brain has been observed, particularly in the naturally occurring obese yellow (Ay/a) mutant mouse. This mouse, as a result thereof, exhibits a late-onset obesity which is not corticosterone dependent. The ectopically-expressed agouti protein antagonizes the MC-3R and MC-4R receptors in the brain mimicking the normal action of AgRP in the hypothalamus. This was corroborated by the generation of an MC-4R knockout mouse that recapitulates the obese phenotype of the agouti mutant mouse (see, Huszar et al., (1997) Cell 88:131-141; and U.S. Pat. No. 5,932,779, issued Aug. 3, 1999).

Interestingly, the subset of hypothalamic arcuate nucleus neurons in which AgRP is expressed also express the orexigenic peptide Neuropeptide Y (NPY). NPY, a 36-residue peptide isolated initially from porcine brain, has also been shown to exhibit potent effects on feeding and body weight gain; see, (Tatemoto et al., (1982) Proc. Natl. Acad. Sci. USA 79:25142518; and Tatemoto et al., (1982) Nature 285:417418). NPY effects are mediated through specific NPY receptor subtypes, a number of which have been identified; see, e.g., (Ambikaipakan Balasubramaniam, (1997) Peptides 18(3):445-457). Interestingly, NPY deficient mice were reported to eat and grow normally; (Erickson et al., (1996) Nature 381:415-418). The absence of NPY is seemingly nullified via compensatory mechanisms; see Ambikaipakan Balasubramaniam, supra.

Leptin, a peripheral hormone secreted by adipocytes, reduces the expression of the orexigenic peptides NPY and AgRP and concomitantly increases the neuronal activity of POMC neurons; (Cowley et al., (1999) Neuron 24:155-163). Enhanced neuronal activity of POMC neurons is believed to result in increased release of α-MSH, agonism at MC-3R and MC4R and, hence, modulation of appetite, energy expenditure and feed efficiency. These conclusions were corroborated by studies of MC-3R and MC-4R knockout mice which were found to be obese as a result thereof; see (Chen et al., (2000) Nature Genet. 26:97-102; and Huszar et al., (1997) Cell 88:131-141).

The stomach-derived ghrelin acts on AgRP/NPY neurons of arcuate nucleus to initiate feeding. Genetic data presented herein demonstrate that AgRP and NPY are obligatory mediators of the orexigenic effect of the circulating ghrelin, in support of similar conclusions from pharmacological studies.

It is desirable to discover new drugs capable of regulating energy metabolism and caloric utilization. Of significant import in this effort are means by which to screen for and evaluate various candidate compounds.

The present invention addresses and meets these needs by disclosing AgRP-deficient animal cells (including AgRP/NPY-deficient animal cells), related non-human transgenic embryos, non-human transgenic animals and non-human transgenic littermates which are also AgRP-deficient or NPY/AgRP-deficient. The disclosed methods for evaluating compounds which effect energy metabolism and caloric utilization in AgRP-deficient or NPY/AgRP-deficient animals also advance these needs.

### SUMMARY OF THE INVENTION

The present invention relates to animal cells which are homozygous for an AgRP deficiency due to a disruption in the gene(s) encoding AgRP. To this end, the present invention also relates to non-human transgenic embryos, non-human transgenic animals and non-human transgenic littermates which are AgRP deficient (AgRP null) due to a disruption in the gene(s) encoding AgRP.

The present invention further relates to animal cells, non-human transgenic embryos, non-human transgenic animals and non-human transgenic littermates which are heterozygous for a functional AgRP gene native to that animal.

The present invention also relates in part to animal cells, non-human transgenic embryos and non-human transgenic littermates having a non-native gene encoding an AgRP protein expressed either in the presence or absence of the native (wild type) AgRP. Preferably, the non-native AgRP gene is the human AgRP gene.

The present invention also relates to animal cells, non-human transgenic embryos, non-human transgenic animals and non-human transgenic littermates which are either homozygous, heterozygous or hemizygous for a deletion of at least a portion of the AgRP gene in combination with a homozygous, heterozygous or hemizygous deletion of at least a portion of the NPY gene or another gene shown to be involved in body weight regulation, such as melanin-concentrating hormone (MCH). Therefore, certain aspects of the invention relate to animal cells, non-human transgenic embryos, non-human transgenic animals and non-human transgenic littermates which are AgRP^{-/+}/NPY^{-/-}; AgRP^{-/+}/NPY^{-/+}; AgRP^{-/-}/NPY^{-/+}, as well as hemizygous alternatives in reference to the two separate alleles. An especially preferred aspect of the present invention relates to AgRP^{-/-}/NPY^{-/-} double knockout mice and related animal cells, non-human transgenic embryos, non-human transgenic animals and non-human transgenic littermates.

The transgenic cells and animals of the present invention are useful in the evaluation of the effect of candidate modulators on the activity of the AgRP gene and/or protein or the expression of the AgRP gene and/or protein as concerns the regulation of energy metabolism. Elimination of the endogenous antagonist (AgRP) of melanocortin (MC) receptors (MC-3R and MC-4R) also enables the transgenic cells and animals of the present invention to be used in the selection and evaluation of potential modulators of MC-3R and MC-4R. This assay is grounded in the fact that AgRP, the endogenous antagonist of the MC receptors, is absent. Candidate modulators should, thus, exhibit similar efficacy at lower concentrations. Double knockout mutants (AgRP/NPY null) enable the study of the effect of modulators on pathways that depend on the activity of both AgRP and NPY, such as the modulators of ghrelin pathway.

The present invention also relates to AgRP- and/or NPY/AgRP-based assays to select for modulators of the protein(s) and/or the MC receptors which affect regulation of energy metabolism and/or body weight. Assays may be cell-based assays or may utilize membrane preparations. Modulators identified through the use of such assays can be used in the treatment of any of the various disorders or diseases associated with the malfunctioning of the neuropeptide(s) and/or melanocortin receptor(s). An AgRP, NPY/AgRP antagonist and/or an MC receptor agonist, for instance, may be used in the treatment of obesity. Alternatively, an AgRP or NPY/AgRP agonist and/or an MC receptor antagonist may be used to treat anorexia and related disorders.

The present invention also relates to AgRP- and/or NPY/AgRP-based assays to select for modulators of ghrelin and/or ghrelin receptor which affect regulation of energy metabolism and/or body weight. Assays may be cell-based assays or may utilize membrane preparations. Modulators identified through the use of such assays can be used in the treatment of any of the various disorders or diseases associated with the malfunctioning of the ghrelin and/or ghrelin receptor(s). For example, an AgRP, NPY/AgRP antagonist and/or a ghrelin receptor antagonist, may be used in the treatment of obesity. Alternatively, an AgRP or NPY/AgRP agonist and/or a ghrelin receptor agonist may be used to treat anorexia and related disorders.

Modulation of AgRP, MC-3R, MC-4R, and ghrelin receptor may also be used to treat the following conditions: growth disorders related to reduced GH, IGF1 function, loss of lean body mass as it occurs in the frail elderly, other states that are characterized as resulting from GH deficiency, cancer, cachexia, disorders associated with depression and anxiety, obesity (by reducing appetite, increasing metabolic rate, or reducing carbohydrate craving), diabetes mellitus (by enhancing glucose tolerance, decreasing insulin resistance), hypertension, hyperlipidemia, osteoathritis, cancer, male and female sexual dysfunction (including impotence, loss of libido and erectile dysfunction), modulation of cytokine release, and skin tanning.

As used herein, the term “functional” is used to describe a gene or protein that, when present in a cell or in vitro system, performs normally as if in a native or unaltered condition or environment. Therefore, a gene which is not functional (i.e., “non-functional”, “disrupted”, “altered”, or the like) will encode a protein which does not function as a wild type, native or non-altered protein, or encodes no protein at all. Such a non-functional gene, such as a non-functional AgRP gene, may be the product of a homologous recombination event as described herein, where a non-functional gene is targeted specifically to the region of the target chromosome which contains a functional form of the gene, resulting in a “knockout” of the wild type or native gene.

As used herein with respect to AgRP, a “modulator” is a compound that causes a change in the expression or activity normally attributed to AgRP, or causes a change in the effect of the interaction of AgRP with its receptor(s), or other protein(s), such as an agonist or antagonist.

As used herein with respect to dual modulators of AgRP and NPY, a “modulator” is a compound that causes a change in the expression or activity normally attributed to AgRP and NPY, or causes a change in the effect of the interaction of AgRP and NPY with its receptor(s), or other protein(s), such as an agonist or antagonist.

As used herein with respect to the melanocortin receptors, a “modulator” is a compound that causes a change in the expression or activity normally attributed to MC-3R or MC-4R, or alters the interaction of MC-3R or MC-4R with its ligand(s), or other protein(s), such as an agonist or antagonist.

As used herein with respect to the ghrelin and/or ghrelin receptor, a “modulator” is a compound that causes a change in the expression or activity normally attributed to ghrelin and/or ghrelin receptor, or alters the interaction of ghrelin with its receptor, such as an agonist or antagonist.

As used herein in reference to transgenic animals of this invention, a “transgene” is a genetic construct including a gene. The transgene is integrated into one or more chromosomes in the cells in an animal by methods known in the art. Once integrated, the transgene is carried in at least one place in the chromosomes of a transgenic animal. A gene is a nucleotide sequence that encodes a protein, or structural RNA. The gene and/or transgene may also include genetic regulatory elements and/or structural elements known in the art.

As used herein, the term “animal” includes all mammals, except when referring to transgenic animals. In the latter capacity, the term excludes humans. The term “animal” includes an individual animal in all stages of development, including embryonic and fetal stages. A “transgenic animal” is an animal containing one or more cells bearing genetic information received, directly or indirectly, by deliberate genetic manipulation at a subcellular level, such as by microinjection or infection with recombinant virus. This introduced DNA molecule can be integrated within a chromosome, or it can be extra-chromosomally replicating DNA. Unless otherwise noted or understood from the context of the description of an animal, the term “transgenic animal” as used herein refers to a transgenic animal in which the genetic information was introduced into a germ line cell, thereby conferring the ability to transfer the information to offspring. If offspring in fact possess some or all of the genetic information, then they, too, are transgenic animals. The genetic information is typically provided in the form of a transgene.

As used herein, a “targeted gene” or “knock out” (KO) is a DNA sequence introduced into the germline of a non-human animal by way of human intervention, including but not limited to, the methods described herein. The targeted genes of the invention include nucleic acid sequences which are designed to specifically alter cognate endogenous alleles, especially endogenous alleles which encode AgRP, or alternatively, both NPY and AgRP. The “knock out” can be the result of an altered, or preferably, completely deleted AgRP gene, but also includes but is not limited to AgRP (and NPY) gene deletions, gene modifications and/or gene insertions which render the native gene nonfunctional or at least substantially nonfunctional or can lead to an AgRP protein (or NPY and AgRP proteins) with altered expression or activity. As noted above, a non-human transgenic animal without an AgRP gene can be used to evaluate the role of AgRP in energy metabolism and its associated disorders, while a NPY/AgRP dual knock out can be used to evaluate the role of NPY/AgRP dual modulators in obesity and other disorders described herein.

As used herein, “AgRP” or “Agrp” refers to agouti-related protein.

As used herein, “NPY” refers to Neuropeptide Y. As used herein, “MC-1R” refers to the melanocortin-1 receptor.

As used herein, “MC-3R” refers to the melanocortin-3 receptor.

As used herein, “MC4R” refers to the melanocortin-4 receptor.

As used herein, “Ghrelin-R” refers to the ghrelin receptor.

As used herein, “MCH” refers to melanin-concentrating hormone.

### BRIEF DESCRIPTION OF THE DRAWINGS

FIG. 1 shows the nucleotide sequence which encodes murine AgRP (SEQ ID NO:1). The murine AgRP gene is disclosed in FIG. 1, SEQ ID NO:1, and was also disclosed previously, see, e.g., GenBank Accession No. U89486). This DNA is that of the preferred target host, Mus musculus (house mouse).

FIG. 2 shows the amino acid sequence of murine AgRP (SEQ ID NO:2).

FIG. 3 shows the nucleotide sequence which encodes human AgRP (SEQ ID NO:3).

FIG. 4 shows the amino acid sequence of human AgRP (SEQ ID NO:4).

FIG. 5 shows the nucleotide sequence which encodes murine NPY (SEQ ID NO:5).

FIG. 6 shows the amino acid sequence of murine NPY (SEQ ID NO:6).

FIG. 7 shows the nucleotide sequence which encodes human NPY (SEQ ID NO:7).

FIG. 8 shows the amino acid sequence of human NPY (SEQ ID NO:8).

FIG. 9 shows schematic diagrams illustrating the murine wild-type Agrp allele, targeting vector, and mutant allele. The mutant allele derives from a double reciprocal homologous recombination between the wild-type allele and targeting vector. R, EcoRI; B, BamHI; K, KpnI; TK, thymidine kinase gene; PGKneo, PGK driven neomycin resistance gene.

FIG. 10 shows a Southern blot analysis of BamHI-digested tail DNAs. The coding probe (shown in FIG. 9) detects an 11 kb restriction fragment from Agrp^{+/+} (wild-type) and Agrp^{+/-} mice, but not from Agrp^{-/-} mice.

FIGS. 11A and B show in situ hybridization with a mixture of two oligonucleotide probes to Agrp. AgRP mRNA is revealed in the arcuate nucleus of wild-type (^{+/+}) mice, but not in the same brain region of Agrp^{-/-} (^{-/-}) mice, respectively.

FIGS. 12A and B show the growth curves of male and female Agrp (wild-type; ^{+/+}; open squares), Agrp^{+/-} (^{+/-}, filled triangles) and Agrp^{-/-} (^{-/-}, open circles) littermate mice (males: ^{+/+}, n=17; ^{+/-}, n=17; n=20; females: +1+, n=17; ^{+/-}, n=21; ^{-/-}, n=23); respectively.

FIG. 13 shows the average daily food intake of 10-week old male Agrp^{+/+} (wild-type), Agrp^{+/-} and Agrp^{+/-} littermate mice (^{+/+}, n=12; ^{+/-}, n=14; ^{-/-}, n=10).

FIG. 14 shows the expression levels of NPY and MCH mRNAs in the hypothalamus of the Agrp^{+/+} (wild-type; ^{+/+}) and Agrp^{-/-} (^{-/-}) littermate mice (n=5 per genotype). *P<0.05, paired student's t test. ARC, arcuate nucleus; LHA, lateral hypothalamic area.

FIGS. 15A and B show the growth curves of male and female wild-type Agrp^{+/+}; Npy^{+/+} (wtwt, open squares); Agrp^{+/+}; Npy^{-/-} (wtko, filled triangles); and Agrp^{-/-}; Npy^{-/-} (koko, open circles) mice (females, n=9-11 per genotype; males, n=10 per genotype). All mice were three months of age at the beginning of the study.

FIGS. 16A and B show cumulative food consumption of the male and female wild-type Agrp^{+/+}; Npy^{+/+} (wtwt, open squares); Agrp^{+/+}; Npy^{-/-} (wtko, filled triangles); and Agrp^{-/-}; Npy^{-/-} (koko, open circles) mice whose growth is summarized in FIGS. 15A and B over the 7 week duration of the study.

FIGS. 17A and B show that Agrp^{-/-}; Npy^{-/-} display a normal hyperphagic response to fasting. Six to 7-month-old female wild-type (wtwt), Agpr^{+/+}; Npy^{-/-} (wtko), and Agrp^{-/-}; Npy^{-/-} (koko) mice (n=14 per genotype) were fasted for 48 hours and then refed with regular chow. Cumulative food intake following refeeding is shown in 7A and changes in body weight during the fasting and refeeding periods is shown in 7B.

FIG. 18 shows the respiratory quotient (RQ) for age-matched male wild-type and Agrp^{-/-} mice at various time periods throughout the day. The ratio of the volume of CO2 produced per kg body weight to the volume of O2 consumed per kg body weight is used to calculate RQ. The daytime difference in RQ (averaged response from 10:30 AM to 5:30 PM) was statistically significant. Values are mean ^{+/-} SEM, n=10 per group. The Agrp^{-/-} mice were found to exhibit a reduced daytime respiratory quotient as compared with the wild-type mice.

FIG. 19 shows the effect of ghrelin on food intake in AgRP/NPY double knockout (koko, n=12) mice and wild-type controls (wtwt, n=12). Relative to saline, intraperitoneal (i.p) injection of 3 μmole/kg human ghrelin stimulated 4-hour food intake in wild-type mice (P<0.001), but was without effect in the double knockout mice.

FIG. 20 shows the structure of the ghrelin peptidomimetic Compound A. FIG. 21 shows the effects of Compound A on food intake of C57B6 mice. Mice were dosed orally (PO) with the ghrelin agonist Compound A and light cycle food intake was measured. All four doses of the compound significantly stimulated 4-hour food intake (P<0.01 versus vehicle). The maximum effect was reached by 3 mg/kg.

FIGS. 22 A and B show the effect of Compound A (3 mg/kg, PO) on food intake. FIG. 22 A shows the effects of Compound A on the 4-hour food intake of wild-type (wtwt, n=12), NPY single knockout mice (wtko, n=11) and AgRP/NPY double null mice (koko, n=12). Compound A significantly stimulated food intake in wild-type mice (P<0.001 versus saline) and NPY single knockout mice (P<0.05 versus saline), but the effect was greatly diminished in the wtko mice. Compound A did not stimulate food intake in the AgRP/NPY double null mice (koko, n=12). FIG. 22 B shows that Compound A significantly stimulates 4-hour food intake in both wild-type (wtwt, n=12, P<0.01 versus saline) and AgRP single knockout mice (kowt, n=10, P<0.001 versus saline).

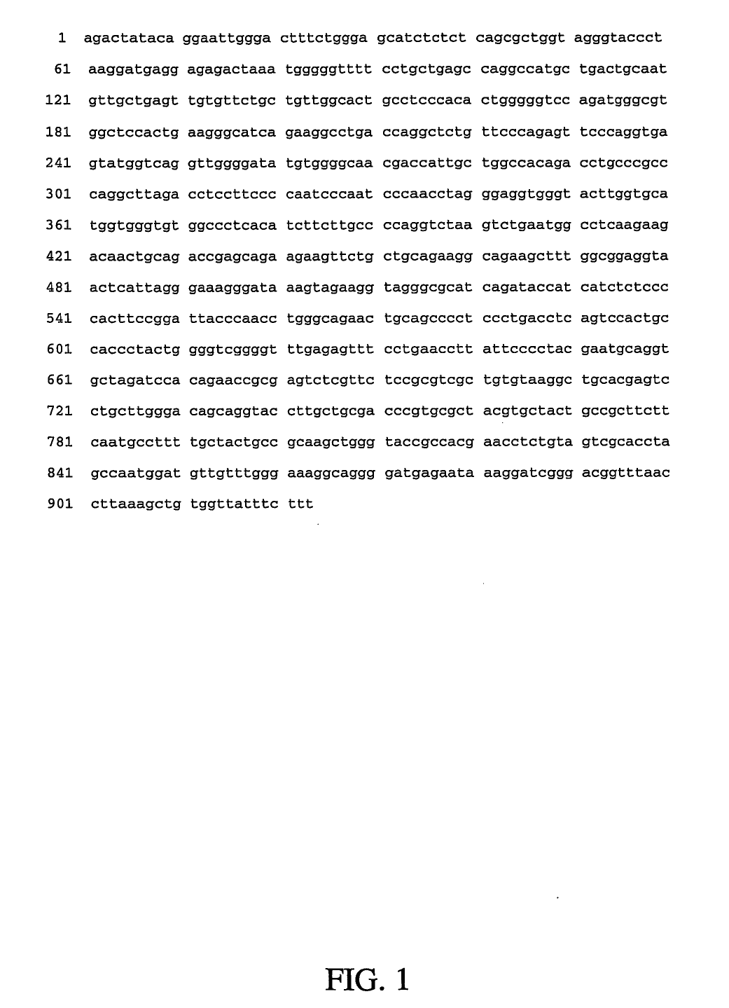

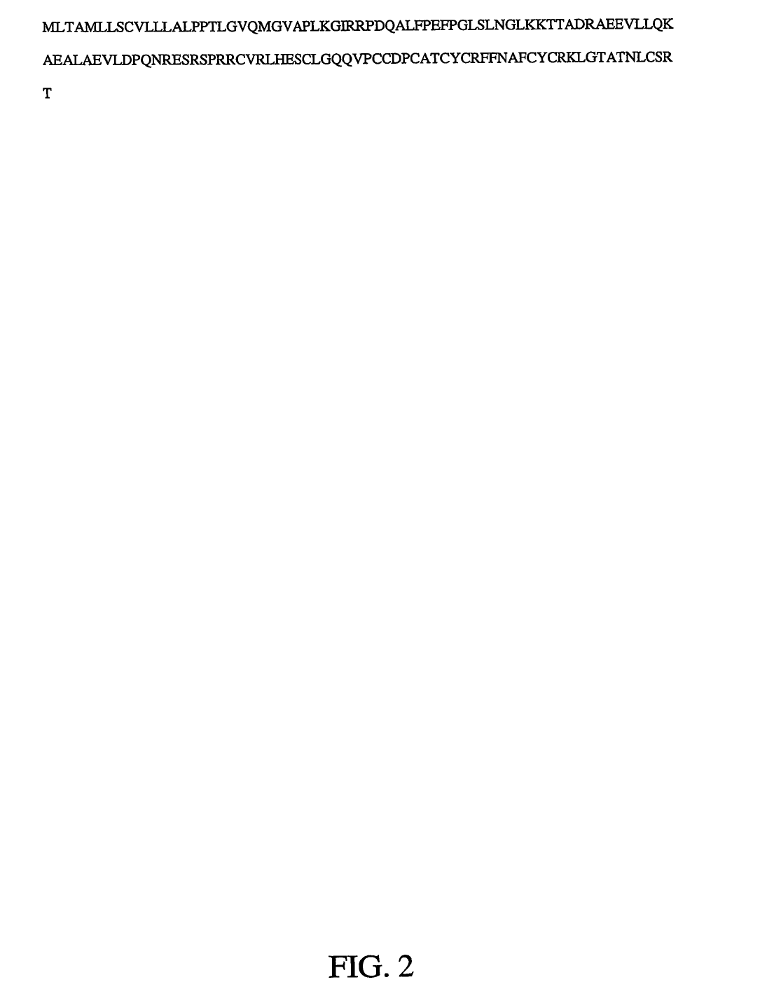

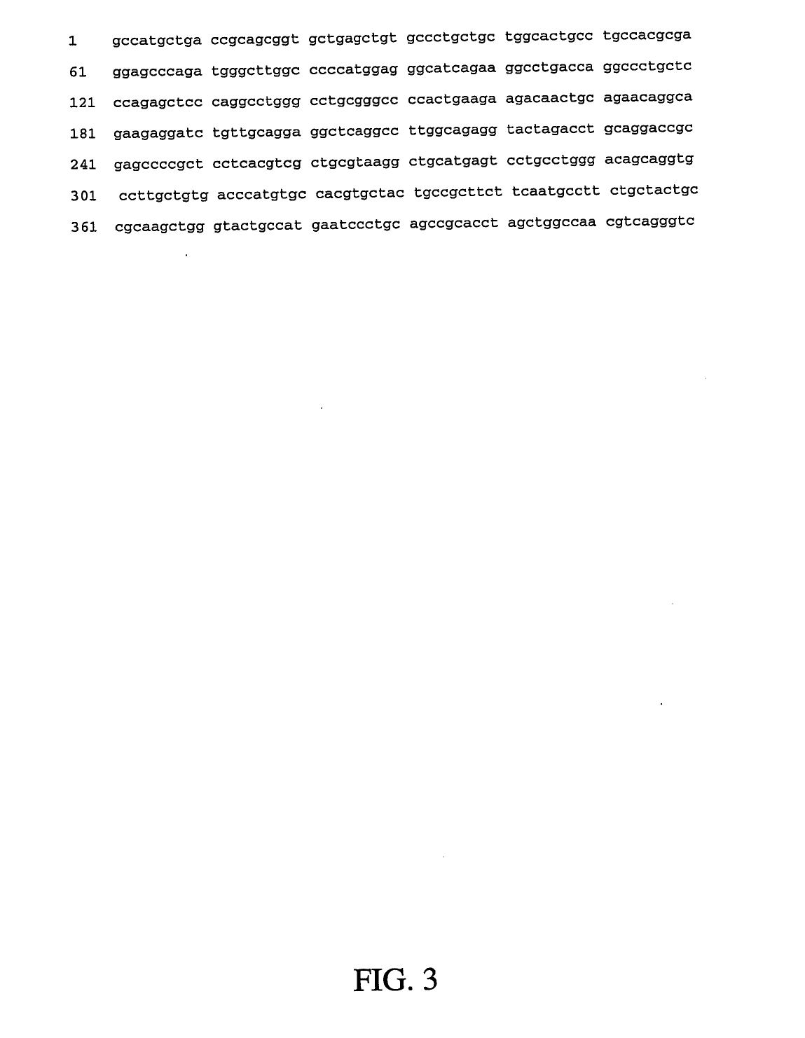

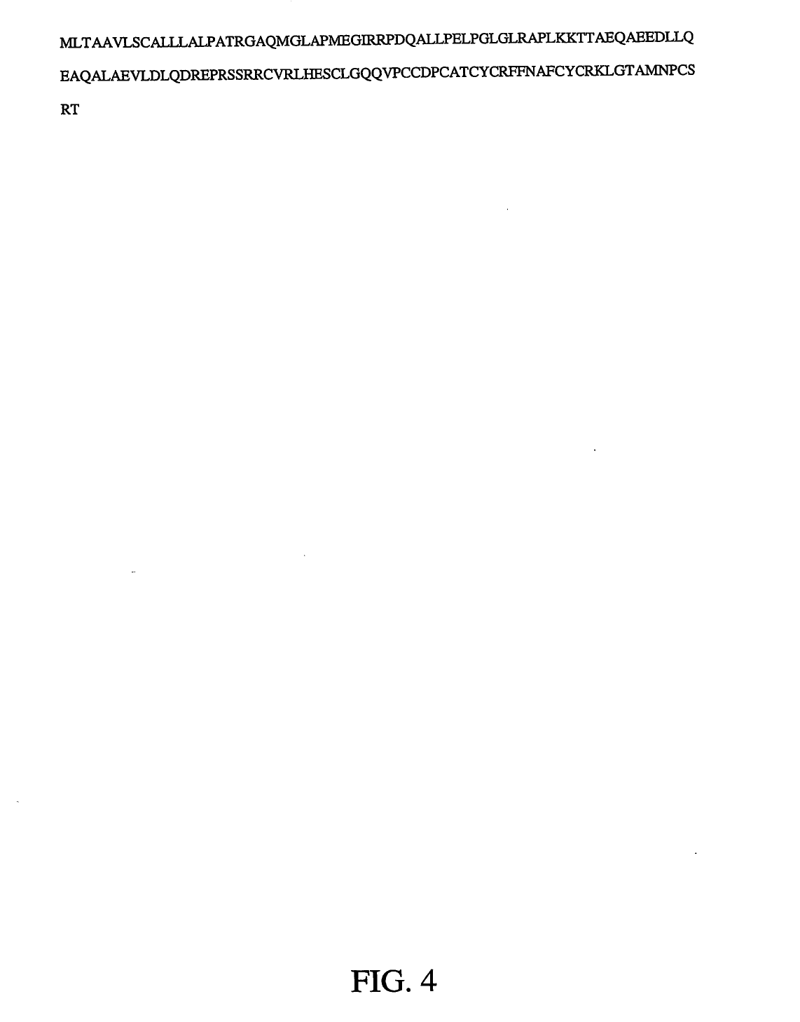

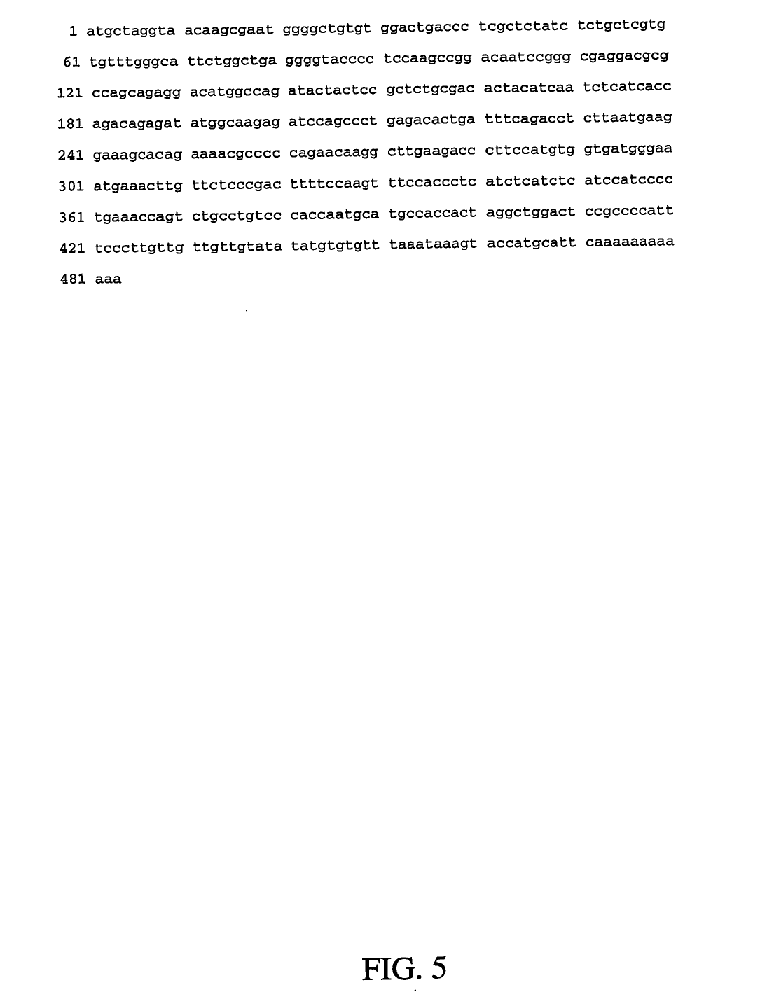

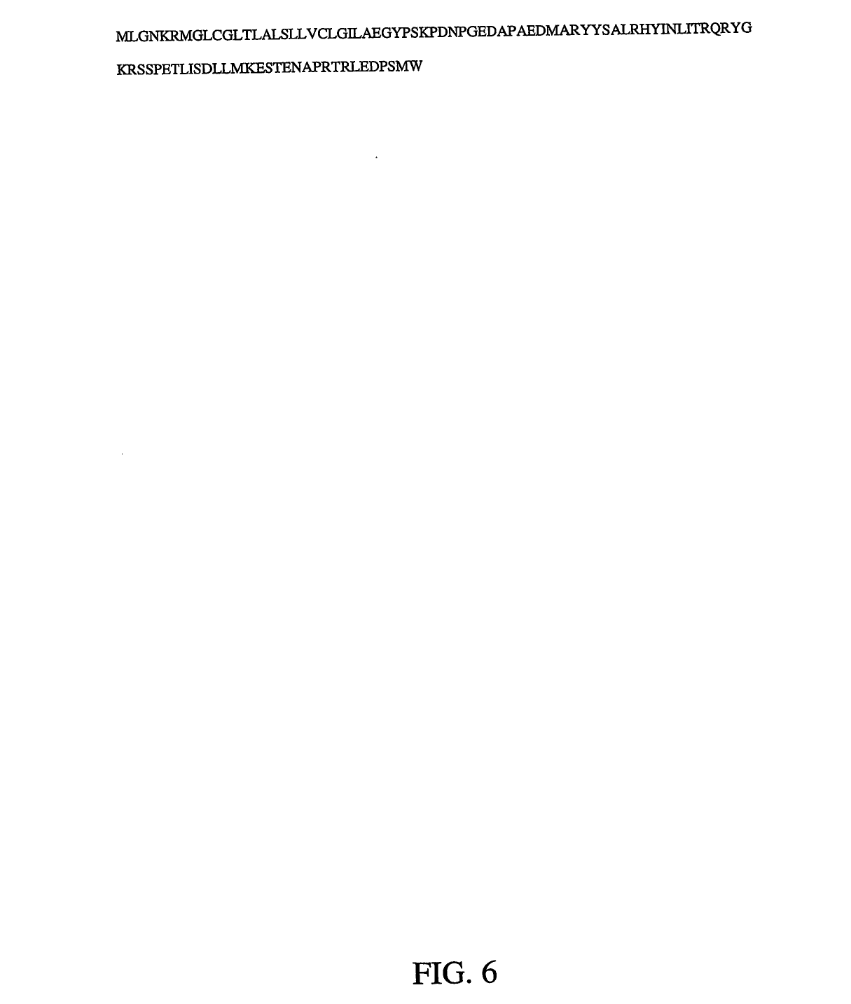

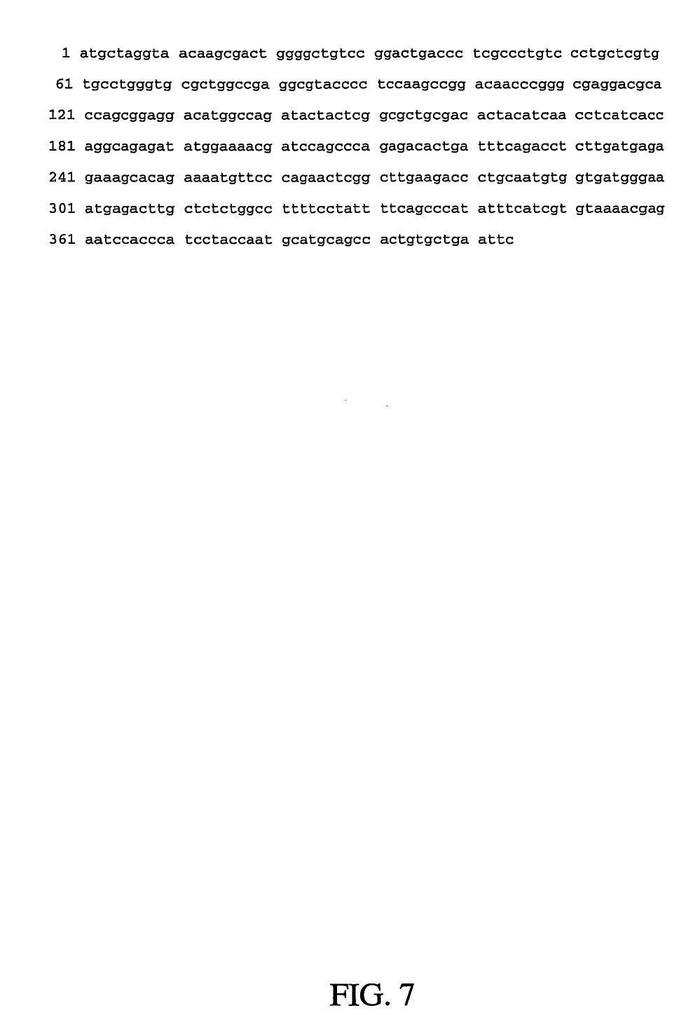

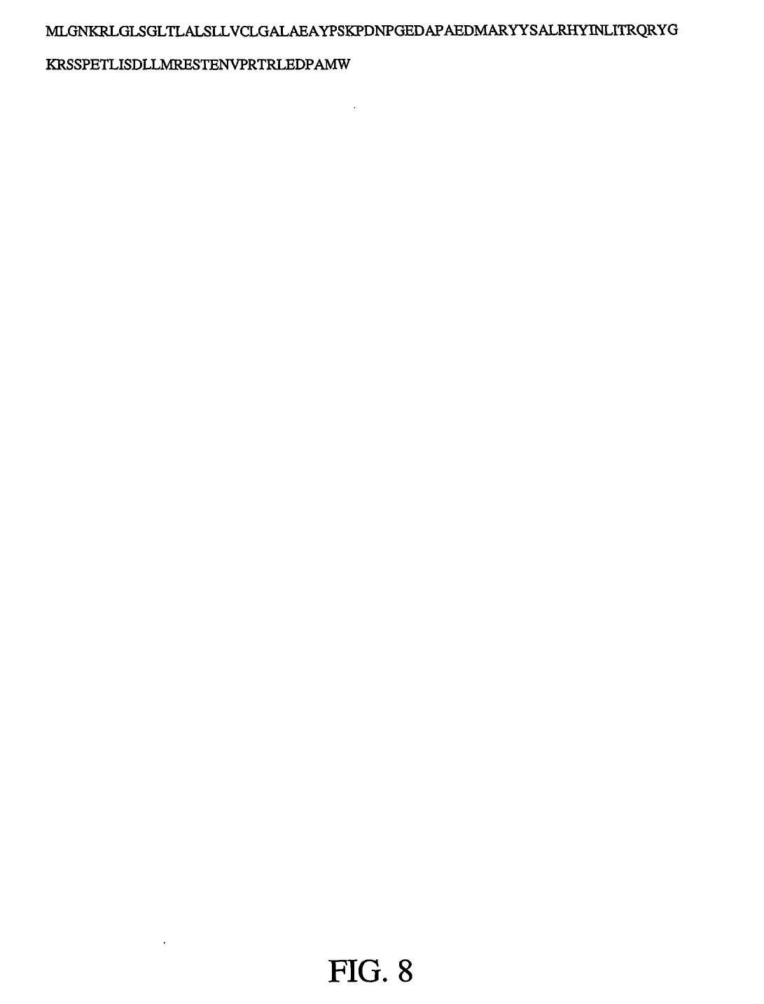

### DETAILED DESCRIPTION OF THE INVENTION

The present invention relates to a transgenic non-human animal lacking native agouti-related protein (AgRP) (AgRP null; AgRP^{-/-}), heterozygous transgenic non-human animals and to transgenic animals having a non-native AgRP protein expressed either in the presence or absence of the native AgRP, as well as AgRP deficient transgenic animals. To this end, the present invention relates to animal cells which are homozygous for an AgRP deficiency due to a disruption in the gene(s) encoding AgRP, as well as to non-human transgenic embryos, non-human transgenic animals and non-human transgenic littermates which are AgRP deficient (AgRP null) due to a disruption in the gene(s) encoding AgRP. The present invention also extends to animal cells, non-human transgenic embryos, non-human transgenic animals and non-human transgenic littermates which are heterozygous for a functional AgRP gene native to that animal. In addition, the present invention relates to animal cells, non-human transgenic embryos and non-human transgenic littermates having a non-native gene encoding an AgRP protein expressed either in the presence or absence of the native (wild type) AgRP. Preferably, the non-native AgRP gene is the human AgRP gene.

The present invention also relates to non-human transgenic embryos, non-human transgenic animals and non-human transgenic littermates which are either homozygous, heterozygous or hemizygous for deletion of at least a portion of the AgRP gene in combination with a homozygous, heterozygous or hemizygous deletion of at least a portion of the NPY gene or another gene shown to be involved in body weight regulation, such as melanin-concentrating hormone (MCH). Therefore, aspects of the invention relate to non-human transgenic embryos, non-human transgenic animals and non-human transgenic littermates which are AgRP^{-/+}/NPY^{-/-}; AgRP^{-/+}/NPY^{-/+}; AgRP^{-/-}/NPY^{-/+}. An especially preferred aspect of the present invention relates to AgRP^{-/-}/NPY^{-/-} double knockout mice and related non-human transgenic embryos, non-human transgenic animals and non-human transgenic littermates.

The generation of AgRP knockout mouse has not been reported and it was not evident that an AgRP knockout mouse would exhibit any phenotype. The essence of the present invention relates to the demonstration that AgRP knockout mice do, in fact, exhibit a notable phenotype, a reduced daytime respiratory quotient (RQ), indicating that these mice preferentially burn fat during the day. This finding, thus, implicates AgRP in the regulation of energy metabolism, particularly in the reduced usage of fat as an energy source, hence, affording the various assays described herein for evaluating and identifying candidate modulators of AgRP which effect energy metabolism and caloric utilization.

The transgenic animals of the present invention can be used in the evaluation of the effect of candidate modulators on the expression and activity of the AgRP gene and/or protein (AgRP and/or NPY gene(s)/protein(s) in the case of the double knockout). The transgenic cells and animals of the present invention can also be used in assays designed to select for and study MC-3R or MC-4R modulators (e.g., agonists), as lack of the endogenous antagonist would predict similar efficacy at lower agonist concentrations. The transgenic cells and animals of the present invention can also be used in assays designed to select for and study ghrelin-R modulators (e.g., antagonists), as AgRP and NPY mediate ghrelin action on feeding stimulation. Accordingly, the transgenic animals of the present invention can be used in the evaluation of the role of AgRP, NPY/AgRP, and/or MC receptor modulators in the regulation of energy metabolism and caloric utilization. Modulators of the neuropeptide(s) and/or melanocortin receptor(s), and ghrelin receptor can be used in the treatment of body weight and muscle mass as defined by lean body mass, including but not limited to disorders such as obesity, diabetes, anorexia, cachexia, male and female sexual dysfunction, growth disorders relating to reduced GH, IGF1 function, loss of lean body mass in the frail elderly, and other states that are characterized as resulting from GH deficiency, cancer, cachexia, and disorders associated with depression and anxiety.

In view of the teachings within this specification, it is within the purview of the artisan of ordinary skill to utilize antisense RNA transgenes, ribozymes or other modulators of RNA expression or other means of modulating AgRP RNA production including promoter mutations, and mutations that affect transcription, to partially or totally knock out expression of the mouse AgRP protein. The antisense transgene used herein would encode a polynucleotide which is at least partially complementary to all or a part of the host AgRP gene and which will hybridize to a target sequence encoded by the host AgRP gene, most specifically a mRNA transcript expressed from the host AgRP gene. Any such oligonucleotide sequence should be at least about 15 to 30 nucleotides in length and preferably more than about 30 nucleotides, wherein this sequence is substantially complementary to the target host gene. The antisense transgene need not be a total complement, but instead should contain adequate sequence identity such that the expressed antisense RNA transgene will effectively hybridize with the expressed mRNA from the host target gene so as to efficiently inhibit concomitant protein expression. These antisense polynucleotides may be produced by subcloning the sequence of interest into an appropriate gene expression vector and transferring this vector to pluripotent embryonic stem cells which may be used as described herein to generate another form of an AgRP deficient non-human transgenic animal.

The generation of AgRP deficient transgenic non-human animals, including mice, aids in defining the in vivo function(s) of AgRP, especially as related to the regulation of energy metabolism and caloric utilization, as well as other indications listed herein, including but not limited to obesity (by antagonizing the effects—reduction of appetite, increase of metabolic rate, reduction of fat intake, or reduction of carbohydrate craving—observed upon proper functioning of the MC receptors), diabetes mellitus (by antagonizing the effects—enhancement of glucose tolerance, decrease of insulin resistance—observed upon proper functioning of the MC receptors), hypertension, hyperlipidemia, anxiety, male and female sexual dysfunction (including impotence, loss of libido and erectile dysfunction), modulation of cytokine release, and skin tanning. Additionally, AgRP null animals can be used as a strain for the insertion of human AgRP genes, and provides an animal model useful in the design and assessment of various approaches to modulating AgRP activity and expression. Such modified transgenic non-human animals can also be used as a source of cells for cell culture. These cells can be used for corresponding in vitro studies of AgRP expression, activity and the modulation thereof.

An aspect of this invention is a method to obtain an animal in which the cells lack a functional AgRP gene native to the animal. By “native gene”, Applicants refer to an AgRP gene that naturally occurs in the animal. If the gene is not mutant, it may also be referred to as wild-type. An altered AgRP gene should not fully encode the same AgRP as native to the host animal, and its expression product can be altered to a minor or greater degree, or absent altogether. In cases where it is useful to express a non-native AgRP gene in a transgenic animal in the absence of a native AgRP gene we prefer that the altered AgRP gene induce a null knockout phenotype in the animal. However a more modestly modified AgRP gene can also be useful and is within the scope of the present invention. The AgRP mutation may be a targeted deletion mutation, a targeted substitution mutation and/or a targeted insertion mutation. However, the preferred mutation is a deletion mutation, and especially preferred is a deletion mutation which results in a deletion of most if not all of the AgRP gene.

The gene encoding agouti-related protein has three coding exons (exons 2, 3, and 4) and one variably spliced non-coding exon (exon 1). The encoded protein is a 131 amino acid protein (132 in the case of the human protein) with a cysteine rich carboxyl terminus believed important to proper functioning as an antagonist of α-MSH. The murine sequence is about 81% identical to the human AgRP polypeptide sequence.

A preferred deletion mutation may contain a deletion of anywhere from 1 nucleotide to deletion of the entire gene, including the open reading frame and associated cis-acting regulatory sequences associated with wild type AgRP. A smaller deletion within the open reading frame is preferably not divisible by three, so as to result in a frameshift mutation resulting in a protein which most likely is non-functional. It is preferred that any such smaller deletion not divisible by three be targeted toward the 5′ region of the open reading frame to increase the possibility of generating a non-functional truncated protein product. However, as noted above, it is preferable that the deletion mutation encompasses most if not all of the AgRP gene so as to ensure prevention of expression of a functional AgRP protein.

The AgRP knock out targeting vector may be generated by methods known in the art. A mouse genomic DNA library was screened in the instant examples with a murine AgRP probe. Mouse genomic clones spanning 23 kb kb were isolated and a gene targeting vector consisting of an 5.2 kb 5′ sequence (long arm) and an 2.2 kb 3′ sequence (short arm) with the pgk-neo gene for positive selection and HSV-tk gene for negative selection was constructed. This construct was linearized and electroporated into AB2.2 ES cells and cultured with G418/FIAU for positive and negative selections. Ten positive clones were selected for expansion and microinjection into blastocysts to generate chimeric mice.

The generalized overall method for obtaining an animal in which the cells lack a functional AgRP gene native to the animal includes inserting an altered AgRP at the place of the native AgRP gene or at another chromosomal location. The transgene can be introduced into embryonic stem cells. ES cells can be obtained from pre-implantation embryos cultured in vitro and fused with embryos (Evans et al., (1981) Nature 292: 154-156; Bradley et al., (1984) Nature 309: 255-258; Gossler et al., (1986) Proc. Natl. Acad. Sci. USA 83: 9065-9069; and Robertson et al., (1986) Nature 322: 445448). Transgenes can be efficiently introduced into the ES cells by a variety of standard techniques such as electroporation, lipofection, DNA transfection, microinjection, or by retrovirus-mediated transduction. The resultant transformed ES cells can thereafter be injected into blastocysts which are then implanted into pseudopregnant animals. The introduced ES cells thereafter colonize and contribute to the germ line of the resulting chimeric animal (Jaenisch, (1988) Science 240: 1468-1474). In alternate embodiments, the transgene-targeted embryonic stem cells can be co-incubated with fertilized eggs or morulae followed by implantation into females.

After gestation, the animals obtained are chimeric founder transgenic animals. The founder animals can then be used in further embodiments to cross with wild-type animals to produce F1 animals heterozygous for the altered AgRP gene. In further embodiments, these heterozygous animals can be interbred to obtain the viable transgenic embryos whose somatic and germ cells are homozygous for the altered AgRP gene and thereby lack a functional AgRP gene. In other embodiments, the heterozygous animals can be used to produce cell lines. In preferred embodiments, the animals are mice.

The use of gene-targeted ES cells in the generation of gene-targeted transgenic mice was described in 1987 (Thomas et al., (1987) Cell 51:503-512), and is reviewed elsewhere (Frohman et al., (1989) Cell 56:145-147; Capecchi, (1989) Trends in Genet. 5:70-76; Baribault et al., (1989) Mol. Biol. Med. 6:481-492; Wagner, (1990) EMBO J. 9:3025-3032; Bradley et al., (1992) Bio/Technology 10:534-539). See also, U.S. Pat. No. 5,464,764, issued to Cappecchi and Thomas on Nov. 7, 1995; U.S. Pat. No. 5,789,215, issued to Berns et al on Aug. 4, 1998, both of which are hereby incorporated by reference). Therefore, techniques are available in the art to generate the AgRP deficient animal cells, non-human transgenic embryos, non-human transgenic animals and non-human transgenic littermates of the present invention. The methods for evaluating the targeted recombination events as well as the resulting knockout mice are also readily available and known in the art. Such methods include, but are not limited to DNA (Southern) hybridization to detect the targeted allele, polymerase chain reaction (PCR), polyacrylamide gel electrophoresis (PAGE), in situ hybridization and Western blots to detect DNA, RNA and protein.

A further aspect of the present invention is a transgenic non-human animal which expresses a non-native AgRP on a native AgRP null background. In particular embodiments, the null background is generated by producing an animal with an altered native AgRP gene that is non-functional, i.e. a knockout. The animal can be heterozygous (i.e., having a different allelic representation of a gene on each of a pair of chromosomes of a diploid genome), homozygous (i.e., having the same representation of a gene on each of a pair of chromosomes of a diploid genome) for the altered AgRP gene, hemizygous (i.e., having a gene represented on only one of a pair of chromosomes of a diploid genome), or homozygous for the non-native AgRP gene. In preferred embodiments, the animal is a mouse. In particular embodiments the non-native AgRP gene can be a wild-type or mutant allele, preferably a wild-type or mutant human allele. In further embodiments the non-native AgRP gene is operably linked to a promoter. As used herein, operably linked is used to denote a functional connection between two elements whose orientation relevant to one another can vary. In this particular case, it is understood in the art that a promoter can be operably linked to the coding sequence of a gene to direct the expression of the coding sequence while placed at various distances from the coding sequence in a genetic construct. Further embodiments are cell lines and cells derived from animals of this aspect of the invention.

Transgenic animals characterized as having a transgene including a non-native AgRP gene on a native AgRP null background, thus, form an aspect of the present invention as are methods of generating same. One method disclosed herein includes providing transgenic animals whose cells are heterozygous for a native gene encoding a functional AgRP protein and an altered native AgRP gene. These animals are crossed with transgenic animals of this invention which are hemizygous for a transgene including a non-native AgRP gene to obtain animals that are both heterozygous for an altered native AgRP gene and hemizygous for a non-native AgRP gene. The latter animals are interbred to obtain animals that are homozygous or hemizygous for the non-native AgRP and are homozygous or heterozygous for the altered native AgRP gene. In particular embodiments, cell lines are produced which form an aspect of the present invention as are cells isolated from any of the animals produced in the steps of the above disclosed method.

The transgenic animals and cells of this invention are useful in the determination of the in vivo functioning of a non-native AgRP in the regulation of metabolism and, ultimately, body weight. The animals are also useful in determining the ability of various forms of wild-type and mutant alleles of a non-native AgRP to rescue the native AgRP null deficiency. The animals are also useful for identifying and studying the ability of a variety of compounds to act as modulators of the expression or activity of a non-native AgRP in vivo, or by providing cells for culture, for in vitro studies.

The non-human transgenic animals of the present invention include non-human mammalian species, including but not limited to transgenic mice, transgenic rats, transgenic guinea pigs, transgenic rabbits, transgenic goats, transgenic non-human primates, such as chimpanzees, rhesus monkeys and green African monkeys, and transgenic cattle. Transgenic mice are preferred and exemplified herein.

A particular aspect of the present invention relates to the analysis permitted hereby of the complex function(s) of AgRP as related to energy metabolism and its associated conditions (s) by generating knockout transgenic mice and studying how various potential modulators interact within these manipulated animals. As described herein in more detail, the native wild type gene is selectively inactivated in totipotent ES cells and used to generate the transgenic mice of the present invention. Techniques are available to inactivate or alter any genetic region to any mutation desired by using targeted homologous recombination to insert specific changes into chromosomal alleles. It has not been known to date whether a mouse AgRP knock out could be produced. Therefore, the present invention relates to diploid animal cells, non-human transgenic embryos, non-human transgenic animals and non-human transgenic littermates which are heterozygous or homozygous for a disrupted AgRP gene resulting in deficient production of the AgRP protein. The cells, embryos and non-human transgenic animals contain two chromosome alleles for AgRP wherein at least one of the AgRP alleles is mutated such that less than wild-type levels of AgRP activity are produced. The diploid mouse cell, embryo or non-human transgenic mouse homozygous for a disrupted AgRP gene may show at least from about 50% to about 100% reduction in AgRP activity compared to a wild type diploid cell. The diploid mouse cell, embryo or non-human transgenic mouse heterozygous for a disrupted AgRP gene may show at least from about 10% to about 100% reduction in AgRP activity compared to a wild type diploid cell. It is within the purview of the artisan of ordinary skill to use known molecular biology techniques to measure the level of transcription, expression and/or functional AgRP activity in mouse cells homozygous, heterozygous or hemizygous for a mutated AgRP gene. Therefore, the present invention especially relates to analysis of the complex function(s) of AgRP as related to energy metabolism and caloric utilization by generating homozygous, heterozygous or hemizygous transgenic mice and studying how various potential modulators interact within these manipulated animals.

In a preferred embodiment, an assay is performed by providing a transgenic animal in accordance with the description herein, exposing the animal to a candidate compound, and measuring the effect of said candidate compound on overall energy metabolism and other related biochemical and physiological responses. Various measures of these responses (e.g., of energy expenditure and/or utilization) are well known and appreciated in the art. Illustrated herein for purposes of exemplification is a measure of respiratory quotient. However, any other measure available in the art that is capable of detecting an effect on energy metabolism and/or other related biochemical and physiological responses is deemed suitable for use in the assays described herein. Metabolic rate is one such example. Metabolic rate (kcal/h) can be calculated from the following equation: (3.815+1.232×RER)×vO₂, where RER is the respiratory exchange ratio [volume of CO₂ produced (ml/kg body weight/h) per volume of O₂ consumed (ml/kg body weight/h)] and vO₂ is the volume consumed per hour. For purposes of measuring the effect of various feeds on the results obtained, the diets of the tested mice can be altered. Preferred embodiments have at least one of the tested animals on a high fat (e.g., Bioserv F3282, 54% kcal from fat; from Bioserve, Frenchtown, N.J.) or medium to high fat (e.g., D12266B, 32% kcal from fat; from Research Diets, New Brunswick, N.J.) diet.

Measurements obtained can be compared to measurements in a genetically similar or identical animal that has not been exposed to the compound. One way to facilitate such measurements would be to feed both AgRP knockout and wild-type mice a high fat diet to promote obesity (diet induced obesity [DIO]). After becoming obese, effects of potential AgRP antagonists (perhaps even in combination with appetite-suppressing agents) on energy metabolism or caloric utilization may be measured in wild-type mice. Any indications as regards the effects of the antagonists on energy metabolism/caloric utilization can be discerned by comparing the results obtained in wild-type mice with that obtained in AgRP knockout mice (which are preferentially oxidizing fat during the day time) and untreated wild-type mice. Similar protocols may be useful in studying the effects of AgRP modulators in other related disorders, such as obesity and diabetes. It will therefore be within the purview of the artisan to utilize the non-human transgenic animals of the present invention to study any number of complex events associated with modulation of AgRP. As additional examples, but in no way presented as limitations, the potential role of AgRP in condition(s) associated with the adrenal gland, e.g., adrenalectomy, hypophysectomy, chronic stress, and hydroxysteroid dehydrogenase inhibitor treatment, may be studied, in light of the fact that AgRP is expressed in the adrenal gland, and the adrenal gland is known to be involved (or associated) with stress, metabolism and steroidogenesis.

Any in vitro or in vivo cell- and/or membrane-based assay described herein (utilizing the AgRP and/or NPY proteins) may be used in conjunction with any of the disclosed transgenic animals, including but not limited to an AgRP^{-/-} knockout mouse and/or a AgRP^{-/-} NPY^{-/-} double knockout mouse to select for these dual modulators, or any other compound which may modulate AgRP and/or NPY, or modulate receptors such as the melanocortin receptors and the ghrelin receptor, which may provide for an improved compound(s) useful in the treatment of the various disorders and diseases disclosed herein. These double knockout mice are also useful to select for modulators (again, agonists or antagonists of AgRP and/or NPY) involved in the regulation of other processes associated with disorders noted herein, which include but are not limited to obesity, diabetes mellitus, hypertension, hyperlipidemia, osteoarthritis, male and female sexual dysfunction (including impotence, loss of libido and erectile dysfunction), skin tanning, anorexia, cachexia, growth disorders relating to reduced GH, IGF1 function, loss of lean body mass as it occurs in the frail elderly, and other states that are characterized as resulting from GH deficiency, cachexia, and disorders associated with depression and anxiety.

It will be within the scope of the invention to submit screened compounds which show an in vitro modulation effect on AgRP to in vivo analysis, preferably by administering the compound of interest to either a transgenic or wild-type animal as described herein to measure in vivo effects of the compound on AgRP and to further measure biological and physiological effects of Compound Administration on the non-human animal. These in vivo studies may be done either alone or in combination with a known MC-3R and/or MC4R ligand, such as but not limited to α-MSH, or the agouti protein.

It is also an essential part of the present invention to measure sensitivity to other neuropeptide-mediated or other pathways that may have been up or down regulated and to measure changes in sensitivity of compounds that modulate these pathways. To this end, testing of compounds that affect MC-3R, MC4R, or other melanocortin receptors, NPY receptors, galanin, ghrelin receptors, MCH receptors, insulin receptors, Orexin receptors, receptors belonging to the bombesin family of receptors (BRS-3, neuromedin receptors, gastrin releasing peptide receptors), motilin receptors, neuromedin U receptors, adrenergic receptors, leptin receptors, modulators of STATs and SOCs transcription factors, phoshpodiesterase enzymes and others are within the scope of uses for the non-human transgenic animals of the present invention, including but not limited to transgenic mice homozygous, heterozygous or hemizygous for an altered native AgRP gene and transgenic mice homozygous, heterozygous or hemizygous for the double knockout of the AgRP and NPY native genes, as described herein and exemplified in the Examples. To this end, a preferred aspect of the present invention relates to the selection of compounds which are shown to modulate either the AgRP and/or NPY proteins, which may be initially identified through in vitro cell and/or membrane based assays. Any such compound may be further studied by administering to a transgenic mouse which has been altered in the AgRP and/or NPY gene(s) and measuring biological characteristics such as disclosed herein.

Compounds may be used alone at appropriate dosages. Alternatively, co-administration or sequential administration of other agents may be desirable.

The present invention also has the objective of providing suitable topical, oral, systemic and parenteral pharmaceutical formulations for use in the novel methods of treatment of the present invention. The compositions containing compounds can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

For combination treatment with more than one active agent, where the active agents are in separate dosage formulations, the active agents can be administered concurrently, or they each can be administered at separately staggered times.

The present invention, therefore, is shown to provide a model system consisting of transgenic animals, especially AgRP^{-/-} mice, cells and assays that are useful in the study of aspects of the etiology of energy metabolism and caloric utilization as related to modulation of AgRP. The various assays are also useful for screening and selecting for compounds that have an effect on overall energy metabolism and caloric utilization, the further study of these compounds and the possible administration of selected compounds to humans in order to regulate disorders which include but are not limited to obesity (by reducing appetite, increasing metabolic rate, reducing fat intake or reducing carbohydrate craving), diabetes mellitus (by enhancing glucose tolerance, decreasing insulin resistance), hypertension, hyperlipidemia, osteoarthritis, male and female sexual dysfunction (including impotence, loss of libido and erectile dysfunction), skin tanning, anorexia, cachexia, growth disorders relating to reduced GH, IGF1 function, loss of lean body mass as it occurs in the frail elderly, and other states that are characterized as resulting from GH deficiency, modulation of cytokine release, and skin tanning. While the preferred subject is a human, other mammals may be an effective host for a compound or compounds identified through the components of the present invention, including but not limited to other mammals, especially mammals of domesticated veterinary use such as canine and feline species, farm animals such as bovine, ovine, porcine, equine, caprine, rodents and additional undomesticated mammals. The finding that AgRP is involved in the regulation of energy metabolism and caloric utilization will allow testing of selected AgRP antagonists for direct measurements of their efficiency to modulate energy metabolism and to treat its associated conditions. AgRP/NPY dual knockout mice can be used to test compounds that affect the activity and/or expression of either AgRP, NPY or both.

The following examples are presented by the way of illustration and, because various other embodiments will be apparent to those in the art, the following is not to be construed as a limitation on the scope of the invention. All animal protocols discussed within the Examples were approved by the Merck Research Laboratories Institutional Animal Care and Use Committee in Rahway, N.J. The mice were housed in microisolator cages (Labproducts™) in a barrier facility with an air shower entrance or in a specific pathogen-free (SPF) facility.

### EXAMPLE 1

### Construction of the AgRP Targeting Vector and Generation of Agtp^{-/-} Mice

A mouse 129SvEv genomic library (Lambda FIX II vector, Stratagene) was screened with a 402 bp mouse Agrp cDNA probe, SEQ ID NO: 9,

catgctgacc gcaatgttgc tgagttgtgt tctgctgttg gcactgcctc ccacactggg
ggtccagatg ggcgtggctc cactgaaggg catcagaagg cctgaccagg ctctgttccc
agagttccca ggtctaagtc tgaatggcct caagaagaca actgcagacc gagcagaaga
agttctgctg cagaaggcag aagctttggc ggaggtgcta gatccacaga accgcgagtc
tcgttctccg cgtcgctgtg taaggctgca cgagtcctgc ttgggacagc aggtaccttg
ctgcgacccg tgcgctacgt gctactgccg cttcttcaat gccttttgct actgccgcaa
gctgggtacg gccacgaacc tctgcagccg cacctagcca at

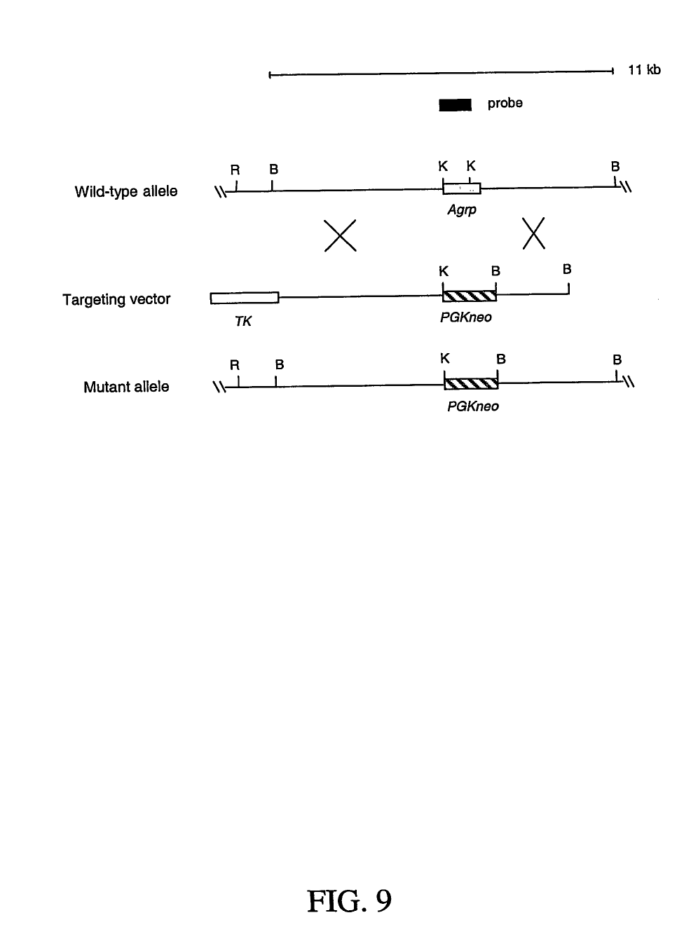

which was generated by PCR using primers derived from the human Agrp; (Shutter et al., (1997) Genes & Dev. 11:593-602). Two positive clones containing 23 kb overlapping mouse Agrp genomic sequences were isolated. Agrp coding and surrounding regions were separately subcloned into pBluescript vector (Stratagene). An Agrp targeting vector was generated by inserting a 5.2 kb 5′ BamHI to KpnI restriction fragment between the Bg1H-KpnI site, and a 2.2 kb 3′ SpeI restriction fragment into the EcoRI sites of pKO Scrambler 1901 (Stratagene), flanking the neomycin resistance cassette. In the targeting vector, a 1.03 kb region of murine Agrp that spans the entire peptide coding sequence was replaced by a neomycin-resistance cassette (FIG. 9). The targeting vector was linearized by SalI digestion and transformed into AB2.2 ES cells (Lexicon Genetics) by electroporation using a BioRad Gene Pulser. Transfected cells were cultured with G418 and FIAU for positive and negative selections, respectively. Approximately 800 clones were selected and ten correctly targeted ES cell clones were identified by Southern blot analysis. Targeted ES clones were injected into C57BL/6 blastocysts, and implanted into pseudopregnant female mice (Gene Targeting: A Practical Approach, (1993), A. L. Joyner (ed.), IRL Press, Papaioannou et al., pp. 107-136). Several chimeric progenies gave germline transmission of the mutant Agrp allele and two independent Agrp^{+/-} lines were established.

### EXAMPLE 2

### Genotyping of the Agrp Mice

To facilitate genotypic identification of the large number of mice generated, three oligonucleotide primers were designed to distinguish the knockout allele from the wild-type allele by PCR. They are as follows:

(SEQ ID NO:12)

| | |
|---|---|
| AG5′: | 5′ - AAA TCA GAA GGC CAC ACC CCG GT -3′; |

(SEQ ID NO:13)

| | |
|---|---|
| AGKO3′: | 5′ - AAA TCG ACC GCG TGG TGG TGC TAA T -3′; |

(SEQ ID NO:14)

| | |
|---|---|
| NEO5′: | 5′ - TAA AGC GCA TGC TCC AGA CTG CCT T -3′; |

Primer pair AG5′ and AGKO3′ generates a 311 bp fragment from the wild-type AgRP allele, while primer pair NEO5′ and AGKO3′ generates a 383 bp fragment from the mutant AgRP allele.

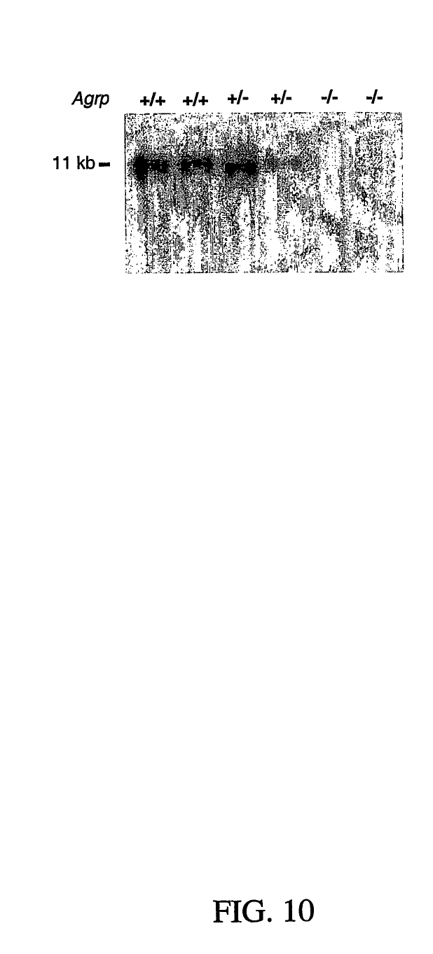

Results from PCR genotyping were confirmed by Southern blot analysis using a 3′ probe and a coding region probe (Molecular Cloning: A Laboratory Manual, 1989, 2^{nd} Edition, Cold Spring Harbour Laboratory Press, Cold Spring Harbour, N.Y., Sambrook et al., pp. 9.31-9.58). The 3′ flanking probe is a 0.75 kb BglII-NotI restriction fragment located 3′ of the short arm of the targeting vector. Upon BamHI digestion, the probe detects a 11.0 kb band from the wild-type Agrp allele, and a 4.0 kb band from the mutant allele. The coding region probe is a 0.78 kb KpnI restriction fragment of Agrp, spanning a region from 53 bp 5′ to the initiation codon to 97 bp 5′ of the stop codon. The coding region probe detects a 11.0 kb wild-type band from BamHI digested genomic DNA, but detects no signal from the mutant allele (FIG. 10).

### EXAMPLE 3

### Production and Characterization of Progeny Mice

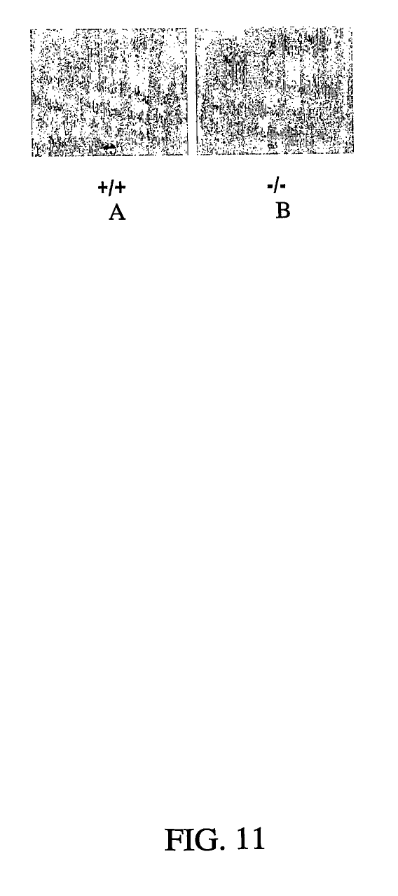

Mating of Agrp^{+/-} mice produced progeny of all three genotypes in the expected Mendelian ratio (Agrp^{+/+} mice, 310; Agrp^{+/-} mice, 662; Agrp^{-/-} mice, 345). Southern blot analysis (FIG. 10) and in situ hybridization (FIG. 11) confirmed the absence of the Agrp gene and mRNA in Agrp^{-/-} mice, respectively. Heterozygous F1 Agrp^{+/-} mice descended from germline transmitting chimeras were interbred to generate F2 Agrp^{+/+} (wild-type), Agrp^{+/-} and Agrp^{-/-} littermate groups for feeding and behavior analysis. All study groups have similar genetic background of 50% C57BL/6; 50% 129SvEv.

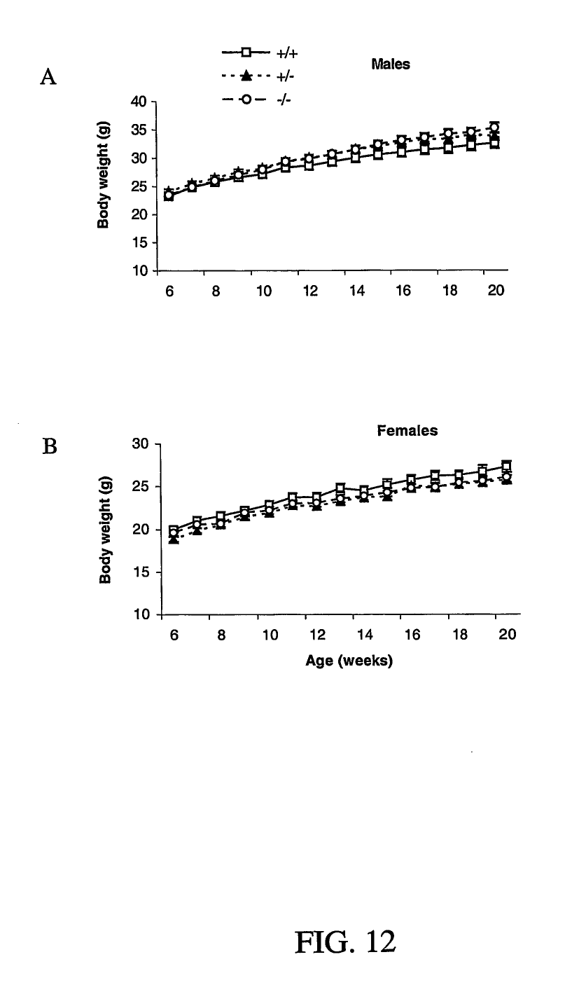

Agrp^{-/-} mice of both genders were fertile and exhibited no gross histological or pathological abnormalities. Group-housed wild-type, Agrp^{+/-} and Agrp^{-/-} littermate mice of both genders exhibited similar growth rates (FIGS. 12A and B). A survey of serum parameters revealed normal levels of plasma glucose, leptin, insulin, triglyceride and free fatty acid in female Agrp^{-/-} mice (Table 1). Mice were fasted for 4 hours prior to blood collection. Numbers are mean ±SEM.

TABLE 1
Serum parameters of 8-month-old female mice

| | Glucose | TG | FFA | Insulin | Leptin |
|---|---|---|---|---|---|
| | (mg/dl) | (mg/dl) | (mM) | (ng/ml) | (ng/ml) |
| Agrp^{+/+} (n = 10) | 118.48 ± 8.80 | 47.69 ± 4.67 | 0.782 ± 0.063 | 0.338 ± 0.057 | 3.125 ± 0.662 |
| Agrp^{-/-} (n = 12) | 126.05 ± 3.36 | 46.62 ± 5.29 | 0.721 ± 0.060 | 0.254 ± 0.038 | 3.764 ± 1.139 |

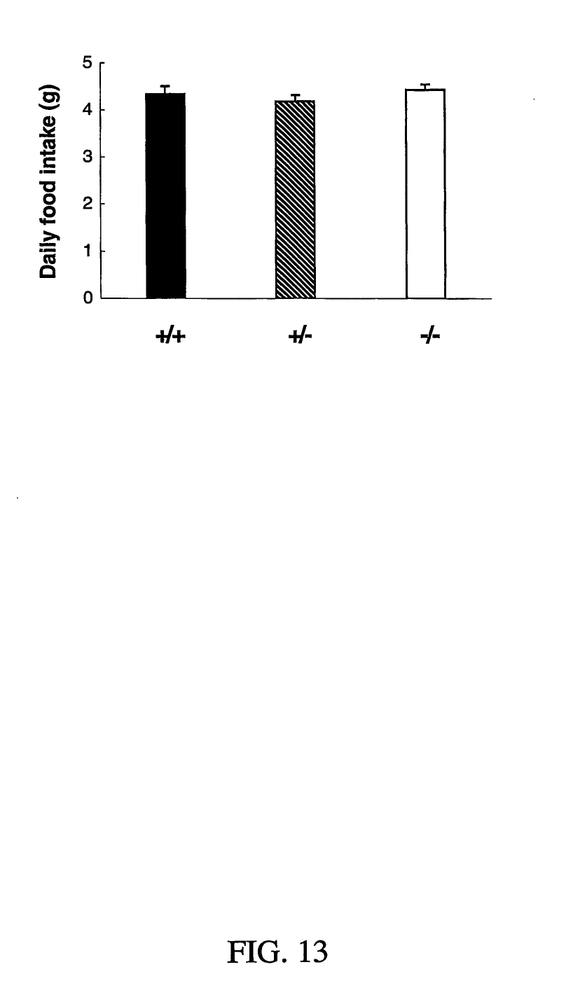

Both male (FIG. 13) and female (data not shown) Agrp^{-/-} mice fed ad libitum had levels of food intake similar to those of wild-type littermate mice. Similarly, male and female Agrp^{-/-} mice did not exhibit any deficits in reflex hyperphagia following 48 hrs of food deprivation (data not shown).

Locomotor activity was examined to evaluate its contribution to total energy expenditure. Agrp^{-/-} mice maintained in a 12-h light/12-h dark environment exhibited normal levels of daily ambulatory activity (distance traveled, Agrp^{-/-} males: 178.1±28.8 m; wild-type males: 126.3±15.6 m, n=8 per genotype, P>0.05; Agrp^{-/-} females: 113.7±26.2 m; wild-type females: 98.9±13.0 m, n=7-8 per genotype, P>0.05) and daily fine motor movements (data not shown).

Core body temperatures were evaluated as a gross measure of metabolic rate. Both male and female Agrp^{-/-} possessed core body temperatures similar to that of wild-type littermate mice (data not shown).

Performance on a rotarod and grip strength were assessed to evaluate motor coordination. Both genders of Agrp^{-/-} and wild-type littermate mice performed comparably on the rotarod and exhibited similar forelimb and hindlimb grip strengths (data not shown).

Conclusions: Agrp^{-/-} mice are viable, and exhibit normal locomotor activity, growth rates, and food intake. Similarly, Agrp^{-/-}; NPY^{-/-} mice are viable and appear normal in feeding behavior or body weight.

### EXAMPLE 4

### In Situ Hybridization

Agrp^{-/-} and age/sex matched wild-type control mice were decapitated under CO₂ anesthesia. Brains were quickly removed and frozen in −40° C. isopentane, and stored at −80° C. until use. Coronal brain sections (14 μM) were cut at −17° C. with a cryostat microtome, and thaw-mounted onto baked microslides. Following fixation in ice-cold 4% phosphate-buffered paraformaldehyde, the tissue sections were stored in 95% ethanol at 4° C. until use. The mouse AgRP hybridization probe consists of an equal molar mixture of two non-overlapping, antisense oligonucleotides against the coding region of AgRP with the following sequences:

(SEQ ID NO: 10)

oligo 296 (mouse AgRP, 45mer, 51% GC): 5′- TGCAGC-
AGAACTTCTTCTGCTCGGTCTGCAGTTGTCTTCTTTGAGG -3′

(SEQ ID NO: 11)

oligo 297 (mouse AgRP, 45mer, 53% GC): 5′- AGCTTG
CGGCAGTAGCAAAAGGCATTGAAGAAGCGGCAGTAGCAC-3′.

The probes were terminally labeled with [α-³³P]dATP and terminal transferase, and hybridization and washing conditions were as described previously (Guan et al., (1998) Mol. Brain Res. 59:273-279).

Quantitative analysis for mRNA levels was carried out as described previously; (Guan et al., (1998) Molec. Brain Res. 59:273-279). Briefly, autoradiographs were captured and analyzed with the MCID/M2 image analyzer (Imaging Research Inc., Ontario, Canada). Density measurement was performed blindly on coded images. Optical density of each region in multiple adjacent brain sections was quantified, and compared against a ¹⁴C-labeled autoradiographic standard (Amersham, Arlington Heights, Ill.), which was calibrated to a series of brain paste containing varying amount of ³³P-dATP to represent tissue mRNA levels (nCi/g brain tissue). An average value for each neuropeptide/receptor at each brain region was then obtained for each mouse, and the results were analyzed statistically by using two-tailed, paired t test.

### EXAMPLE 5

### Evaluation of Appetitive Behavior

Agrp^{+/+}, Agrp^{+/-}, and Agrp^{-/-} mice were individually housed in microisolator cages at approximately one month of age and at least seven days prior to initiation of the experiments. Regular mouse chow (Teklad 7012: 5.67% kcal from fat; 3.41 kcal/g, Harlan Teklad) was provided to 9.5 to 10 week-old male mice as pellet food in wire cage tops containing food hoppers and weighed daily for three days. Food intake is reported as the average food consumed per animal per day over the course of three consecutive days (Agrp^{+/+}, n=12; Agrp^{+/-}, n-10; Agrp^{-/-}, n=12).

### EXAMPLE 6

### Expression Levels of NPY and MCH in Wild Type and Agrp^{-/-} Mice

Compensation by AgRP has been proposed to explain the lack of a feeding phenotype in Npy^{-/-} mice; (Cowley et al., (1999) Neuron 24:155-163; and Marsh et al., (1999) Brain Res. 848:66-77). Evidence supporting this hypothesis included: i) the nearly exclusive coexpression of NPY and AgRP in a class of arcuate nucleus neurons (Broberger et al., (1998) Proc. Natl. Acad. Sci. USA 94:3128-3133; Hahn et al., (1998) Nature Neuroscience 1:271-272; Ollmann et al., (1997) Science 278:135-138; Shutter et al., (1997) Genes Dev. 11:593-602); ii) increases in the levels of both peptides and their corresponding mRNAs in response to starvation; iii) an enhanced sensitivity to centrally administered AgRP83-132 in NpY^{-/-} mice (Marsh et al., (1999) Brain Res. 848:66-77); and iv) the finding that NPY can functionally antagonize signaling by MTII, an agonist of Mc3r and Mc4r (Cowley et al., (1999) Neuron 24:155-163). To test whether AgRP could compensate for the loss of NPY in NPY^{-/-}mice, we generated a strain of Agrp^{-/-}; Npy^{-/-}mice. Agrp^{-/-}; Npy^{-/-}mice are viable and overtly normal without obvious feeding and body weight deficits.

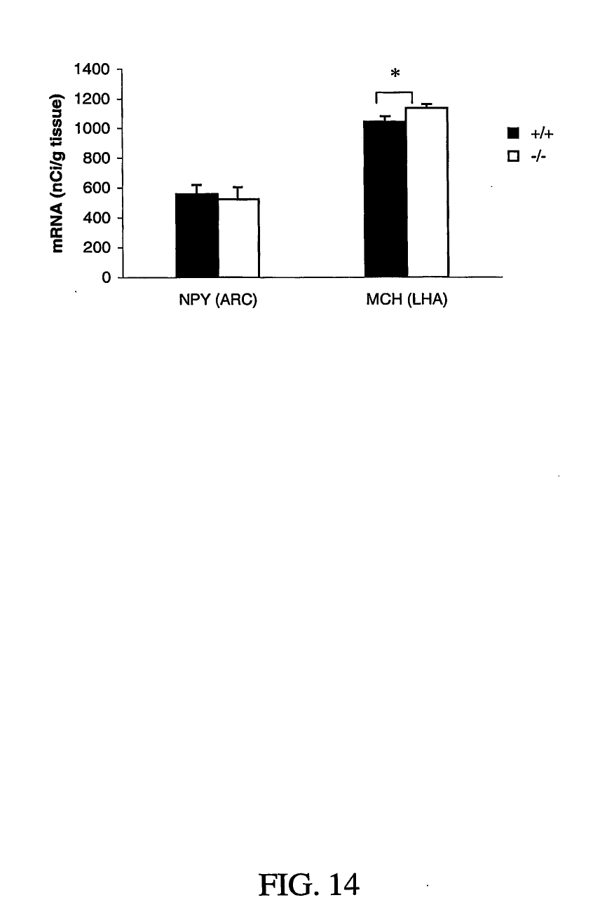

In order to gain insight into the role of NPY and other feeding regulators in AgRP^{-/-} mice, in situ hybridization was performed in order to determine the brain mRNA levels in 5-month-old male animals for NPY, melanin-concentrating hormone (MCH), POMC, MC3r and MC4r. Quantitative analyses revealed no significant changes in the levels of NPY mRNA in AgRP^{-/-} mice (FIG. 14).

However, the data reveal a small (9%), but consistent increase in MCH mRNA (P<0.05; n=5) in the lateral hypothalmic area of AgRP^{-/-} mice (FIG. 14). Although less potent than AgRP and NPY at stimulating feeding, MCH is an orexigenic peptide generally believed to act downstream of AgRP and NPY in the regulation of appetite Although small, the observed increases in MCH mRNA may represent a form of compensation for AgRP deficiency.

Changes in mRNA levels for POMC, MC3r and MC4r were not detected.

### EXAMPLE 7

### Indirect Calorimetry of Wild-Type and Agrp^{-/-} Mice

Respiratory Quotient (RQ) was measured by indirect calorimetry using a 16-chamber open-circuit Oxymax system (Columbus Instruments). Male 16 week-old Agrp^{-/-} (n=10) and Agrp^{+/+} (n=10) littermates were maintained at 21 to 24° C. in a 12-h light-dark cycle (lights on 8 AM to 8 PM) with food (Teklad 7012) and water available ad libitum. Animals were individually housed in specially built plexiglas cages (20×10.5×12 cm) through which room air was passed at a flow rate of 0.53 liter/min. Exhaust air from each chamber was sampled at 15-minute intervals for a period of 75 seconds. Sampled air was sequentially passed through O₂ and CO₂ analyzers (Columbus Instruments) for determination of O₂ and CO₂ content. RQ is the respiratory quotient calculated from the volume of CO₂ produced (ml/kg body weight/h) per volume of O₂ consumed (ml/kg body weight/h). Animals were conditioned to the chambers prior to the study. The response of each animal was recorded on three separate occasions and the average daily RQ response, taken as the average of all readings between 10:30 AM and 5:30 PM, was determined for each animal.

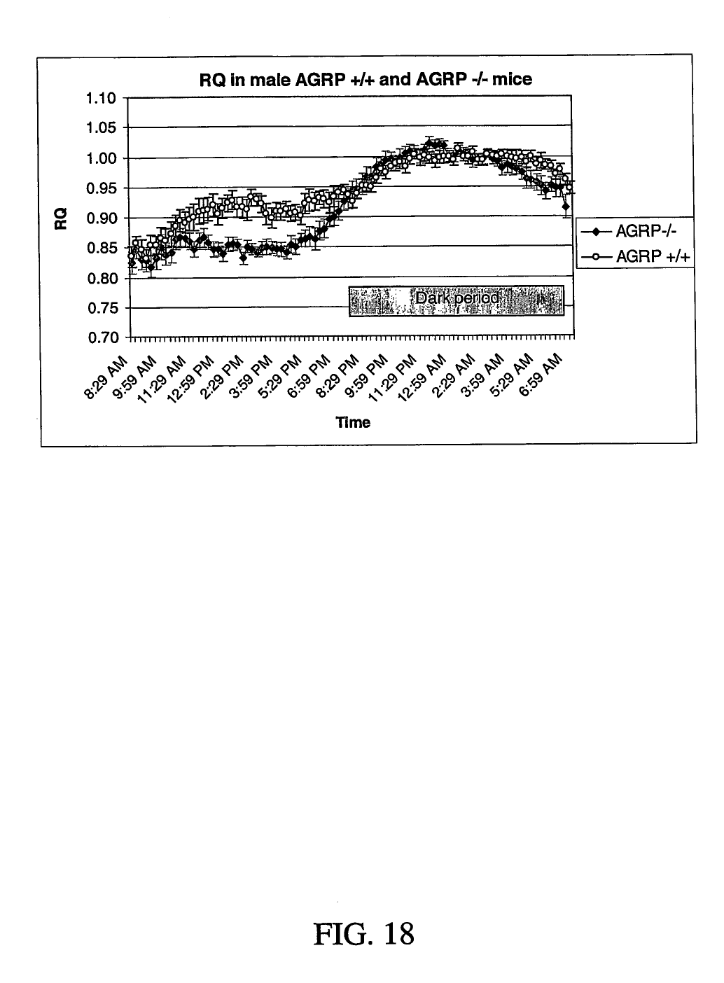

FIG. 18 shows the RQ for wild-type and Agrp^{-/-} mice at various time periods throughout the day. The average daytime RQ response for AgRP^{-/-} mice, 0.85^{+/-}0.03, was significantly different from wild-type mice, 0.91^{+/-}0.04, (P<0.01). The relative utilization of fat versus carbohydrate can be estimated from RQ measurements using the simplified model of carbohydrate and fat oxidation proposed by Elia and Livesey World Rev Nutr Diet (1992) 70:68-131 with the assumption of steady-state protein oxidation proposed by Flatt et al., (1991) J. Nutr Biochem 2:193-202. Using the simplified model, it was determined that AgRP^{-/-} mice derive approximately 20% more of their daytime energy from fat when compared to wild-type control mice. The RQ calculated from respiratory gas exchange supports the findings of the experiment—the Agrp^{-/-} mice exhibit a reduced daytime RQ as compared with the wild-type mice. AgRP does in fact alter RQ. These findings implicate AgRP in the modulation of caloric utilization.

Subtle changes, such as those seen in RQ, suggest that the absence of AgRP does play a real role in energy homeostasis. Thus, acute modulation of AgRP may provide mechanisms for metabolic changes which could result in body weight and adiposity modulation.

### EXAMPLE 8

### Generation and Characterization of Agrp/Npy Double Knockout Mice

To generate Agrp^{-/-}; Npy^{-/-} mice, Agrp^{+/-}; NPY^{+/+} mice were crossed with Agrp^{+/+}; Npy^{-/-} mice. The resulting Agrp^{+/-}; Npy^{+/-} mice were backcrossed with Agrp^{+/+} Npy^{-/-} mice to generate Agrp^{+/-}; Npy^{-/-} mice which were then interbred to produce Agrp^{-/-}; Npy^{-/-} and Agrp^{+/+}; Npy^{-/-} mice. In parallel, Agrp^{+/+}; Npy^{+/-} mice were crossed with Agrp^{+/+}; Npy^{+/+} mice to generate the Agrp^{+/+}; Npy^{+/+} wild-type control mice with similar genetic background. All study groups were of a similar genetic background of 12.5% C57 BL/6; 87.5% 129 sv.

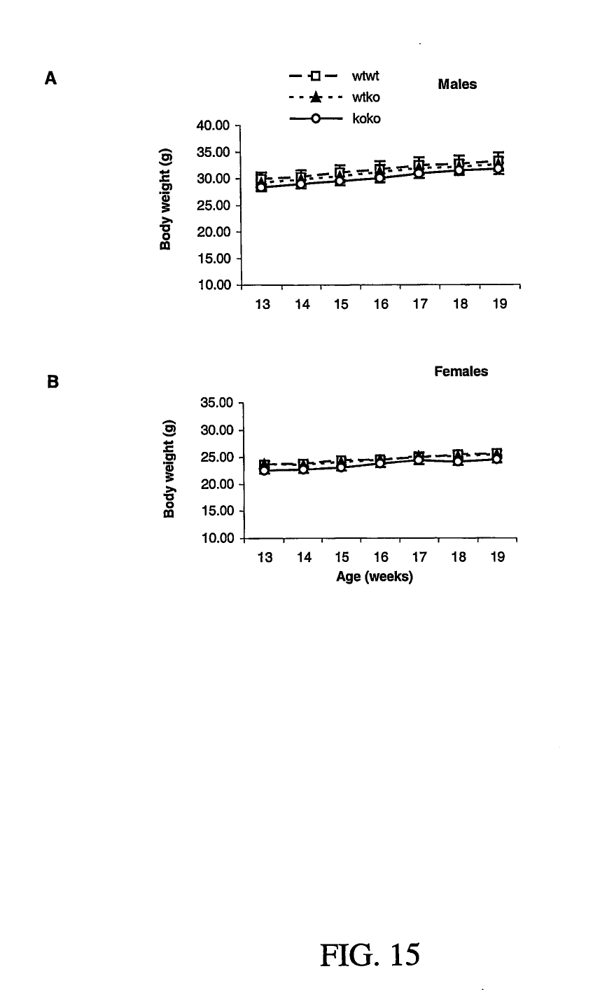

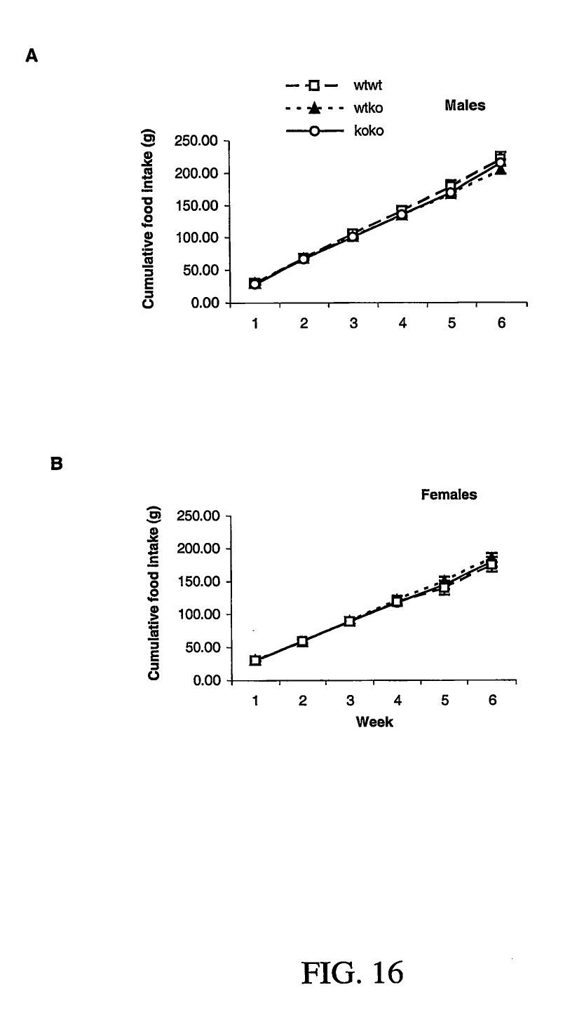

Generally speaking, Agrp^{-/-}; Npy^{-/-} mice are viable and overtly normal without obvious feeding and body weight deficits. This observation is based on growth rate and food intake (consumption) of 3-month-old Agrp^{-/-}; Npy^{-/-} that were maintained on regular chow for a 7-week period. FIG. 15 establishes that the growth rates of male and female mice Agrp^{-/-}; Npy^{-/-} (koko) mice were comparable to those of wild-type and Agrp^{+/+}; Npy^{-/-} (wtko) mice. FIG. 16 establishes that food consumption of the mice was also comparable regardless of their genotype.

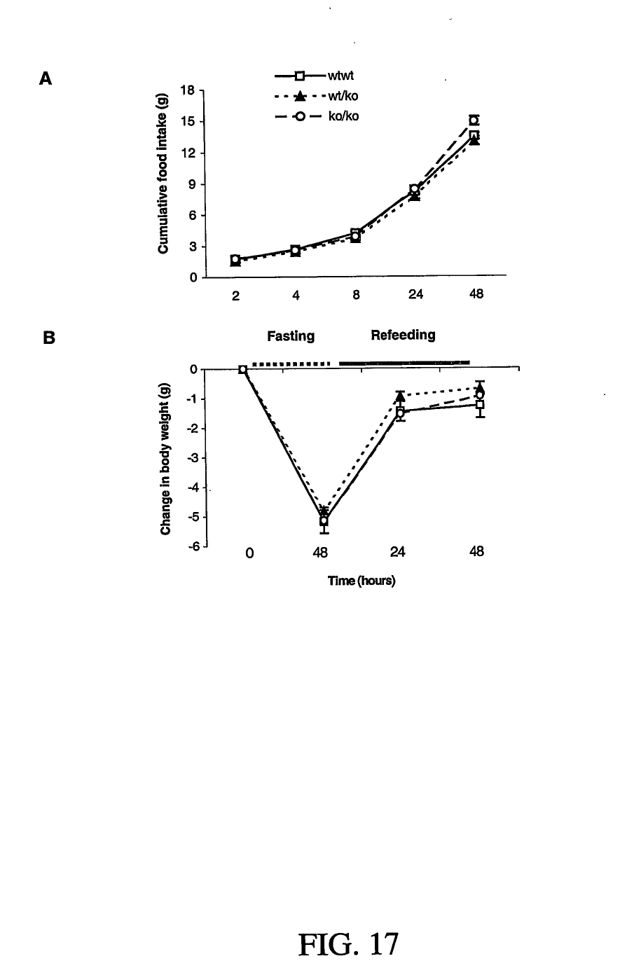

The feeding response of wild-type, Agrp^{+/+}; Npy^{-/-} and Agrp^{-/-}; Npy^{-/-} mice was evaluated after a 48 hour period of food deprivation. Similar to Agrp^{-/-} mice, Agrp^{-/-}; Npy^{-/-} mice mounted a normal hyperphagic response (FIG. 17). Leading to the conclusion that AgRP and NPY can be abolished simultaneously without a detrimental effect on appetitive behavior.

### EXAMPLE 9

### The Effects of Ghrelin on Food Intake of Wild-type and Agrp^{-/-}; Npy^{-/-} Double Knockout Mice

Stomach-derived ghrelin is the first peripheral orexigenic hormone identified (Kojima, et al., (1999) Nature 402: 656-660; Tschop, et al., (2000) Nature 407: 908-913; Nakazato, et al., (2001) Nature 409: 194-198). In addition to its ability to stimulate growth hormone secretion, ghrelin can also stimulate caloric intake and can increase body weight and adiposity. Evidence for its effect on food intake being mediated by neuropeptide Y (NPY) and Agouti-related protein (AgRP) in the CNS has been supported by a number of experimental approaches, including blockade of ghrelin-induced food intake with intracerebroventricular (icv) injection of ghrelin, and antibodies against NPY and AgRP and pharmacological treatment with, NPY Y1 receptor antagonists (Nakazato, et al., (2001) Nature 409: 194-198; Wren, et al., (2000) Endocrinology, 141: 43254328; Kamegai, et al., (2001) Diabetes, 50: 2438-2443; Shitani, et al., (2001) Diabetes 50, 227-232), as well as electrophysiological approaches demonstrating that ghrelin can activate NPY/Agrp neurons and simultaneously reduce the activity of POMC neurons (Cowley, et al. (2003) Neuron, 37: 649-661). To further substantiate the role of AgRP and NPY as mediators of ghrelin's orexigenic effects, we peripherally administered ghrelin in Agrp^{-/-}; Npy^{-/-} double knockout and wild-type control mice.

Native human and rat ghrelin (1-28 with Ser-3 octanoyl group) were custom synthesized by SynPep Corporation, Dublin, Calif. Ghrelin (in 100 μl saline, i.p), Compound A (in 200 μl aqueous solution containing 5% Tween-80 and 0.5% methylcellulose, PO), or vehicle were administered at 10:00 o'clock, and food intake was measured 4 hours later.

Mice were individually housed at approximately one month of age. Regular mouse chow (Teklad 7012: 5.67% kcal from fat; 3.41 kcal/g, Harlan Teklad) was provided as pellet food in wire cage tops containing food hoppers.

Food intake values were reported as means ± standard errors of the means, and analyzed by the two tailed, unpaired Student t test. P values of <0.05 were reported as significant.

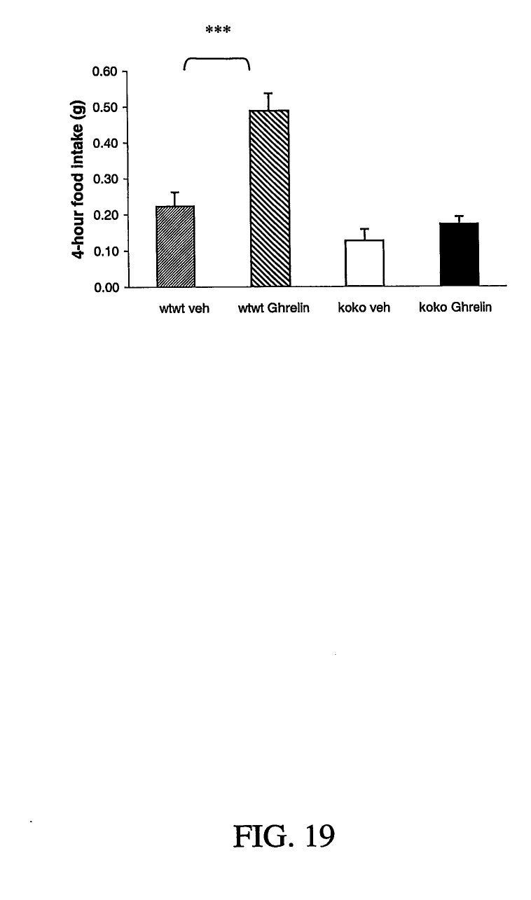

Our dose titration experiment showed that, during light phase, a single intraperitoneal (i.p.) injection of human ghrelin (3 μmole/kg) consistently stimulated 4-hour food intake in satiated wild-type mice. When administered to ad libitum fed satiated Agrp^{-/-}; Npy^{-/-} double knockout mice and wild-type control mice, human ghrelin stimulated 4-hour food intake by 120% in the wild-type controls, but had no effect on food intake of Agrp^{-/-}; Npy^{-/-} double knockout mice (FIG. 19). This result demonstrates that AgRP and NPY are obligatory mediators of the orexigenic effects of circulating ghrelin.

### EXAMPLE 10

### In Vitro Assays of Ghrelin Agonist Compound A

Binding assay: Membrane binding assays were performed on transiently transfected COS-7 cells expressing human ghrelin-R (GHSR1a) from the plasmid vector pCI-neo (Promega, Madison, Wis.) as described (Bednarek, M. A., et al. (2000) J. Med. Chem. 43:4370-4376; Howard, et al (1996) Science 273, 974-977; Pong, S.-S. et al., (1996) Mol. Endocrinol. 10, 57-61). Membranes were prepared by hypotonic lysis, frozen in liquid nitrogen, and stored at −80° C. as described²⁶. Binding buffer contained 25 mM Tris, pH7.4, 10 mM MgCl₂, 2.5 mM EDTA, 0.1% BSA (Sigma, St. Louis, Mo.), and the following protease inhibitors: 4 g/mL leupeptin (Sigma), 40 g/mL bacitracin (Sigma), 5 g/mL aprotinin (Roche Molecular Biochemicals, Indianapolis, Ind.), 0.05 M AEBSF (Roche Molecular Biochemicals), and 5 mM phosphoramidon (Boeringer Mannheim). [³⁵S]MK-0677 (0.05 nM, specific activity ˜1200 Ci/mmol ref. 27) or [His[¹²⁵ I]]-human ghrelin (0.1 nM, specific activity ˜2000 Ci/mmol; NEN Life Sciences, Boston, Mass.) was bound to 4 μg of membrane protein/well with or without competing test ligand. The bound membranes were filtered on 0.5% polyethyleneimine prewet filters (UniFilter 96 GF/C, Packard, Meriden, Conn.). Filters were washed 3 times (50 mM Tris, pH7.4, 10 nM MgCl₂, 2.5 mM EDTA, 0.05% BSA) dried, and counted with Microscint 20 (Packard, Meriden, Conn.).

Specific binding is defined as the difference between total binding and nonspecific binding conducted in the presence of 500 nM unlabeled human ghrelin. IC₅₀ calculations were performed using Prism 3.0 (GraphPad Software, San Diego, Calif.). The IC₅₀ values were measured in triplicates. Aequorin Bioluminescence Assay: A stable cell line expressing the human ghrelin receptor (GHSR1a) and the aequorin reporter protein were used to measure agonist-induced mobilization of intracellular calcium as described (Howard, et al., (1996) Science 273:974-977; Button, D.; Brownstein, M. (1993) Cell Calcium 14:663-671). Functional EC₅₀ values were measured in triplicates.

Rat Pituitary Growth Hormone (GH) Release: Compound functional activity was evaluated by measuring growth hormone secretion from primary cultures of rat anterior pituitary cells (Small, et al., (1998) Regulatory Peptides 75-76 and 301-307). Cells were isolated from rat pituitaries by enzymatic digestion with 250 μg/mL DNAse 1 and 0.5% trypsin in Hanks' balanced salt solution. The cells were suspended in culture medium and adjusted to a concentration of 1.5×10⁵ cells/ml and 1.0 ml of this suspension was placed in each well of a 24-well tray. Cells were maintained in a humidified 5% CO₂/95% air atmosphere at 37° C. for 3-4 days. The culture medium consisted of DMEM containing 0.37% NaHCO₃, 10% horse serum, 2.5% fetal bovine serum, 1% nonessential amino acids, 1% glutamine, 1% nystatin, and 0.1% gentamicin. Before testing compounds for their capacity to inhibit GH release, cells were washed twice 1.5 hr before and once more immediately before the start of the experiment with the above culture medium containing 25 mM Hepes (pH 7.4). Compounds were tested in quadruplicate by adding them in 1 ml of fresh medium to each well and incubating them at 37° C. for 15 minutes followed by centrifugation at 2000×g for 15 min to remove any cellular material. The supernatant fluid was assayed for GH by a double antibody radioimmunoassay. Table 2 provides a comparison of the in vitro binding and functional properties of Compound A relative to the binding and functional properties of ghrelin.

TABLE 2
In vitro and Functional Properties of Ghrelin and Compound A

IC50 or EC50 (nM)

| | Ghrelin | L-166446 |
|---|---|---|
| ¹²⁵I-human ghrelin binding | 0.7 | 0.3 |
| ³⁵S-MK-0677 Binding | 0.3 | 0.1 |
| Aequorin Function | 1.4 | 3.1 |
| Pituitary GH Release | 0.7 | 0.7 |

### EXAMPLE 11

### The Effects of the Ghrelin Agonist Compound A on the Food Intake of Agrp^{-/-} Single Knockout, Npy^{-/-} Single Knockout, and Agrp^{-/-}; Npy^{-/-} Double Knockout Mice

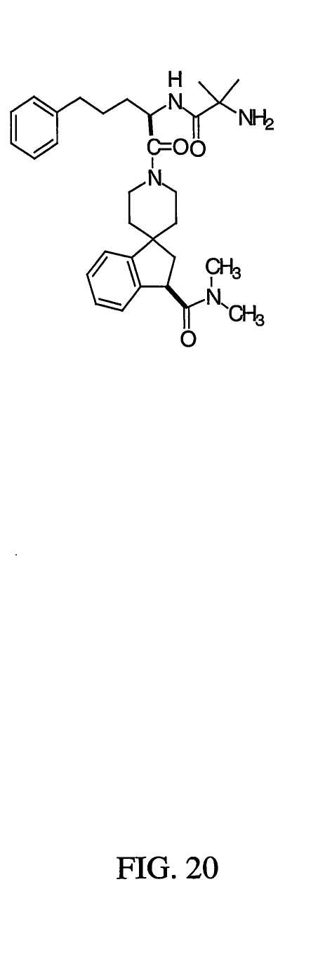

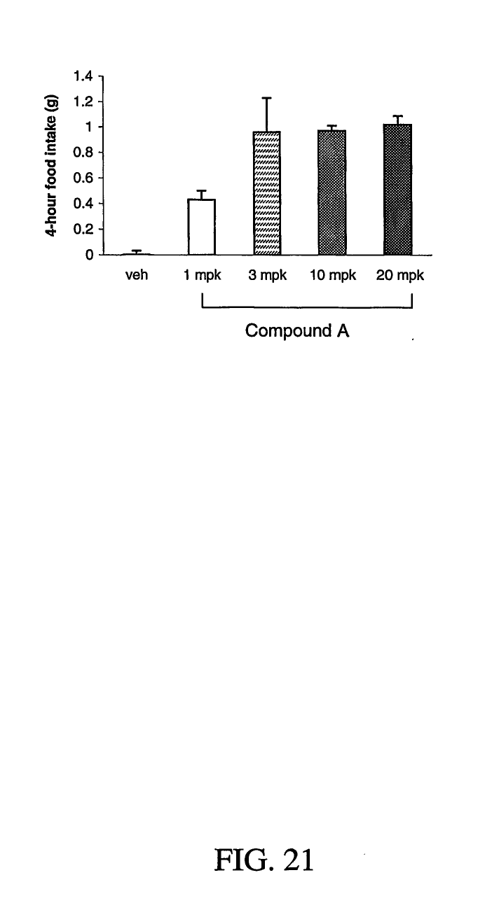

We also evaluated a potent oral ghrelin peptidomimetic Compound A, for its effect on food intake in the wild-type and Agrp^{-/-}; Npy^{-/-} double knockout mice. Compound A (FIG. 20) is a ghrelin agonist (which is generically described in U.S. Pat. No. 5,578,593 the teachings of which are incorporated herein by reference) known to promote the release of growth hormone in humans and animals. Data obtained from an in vitro binding assay demonstrated that Compound A and ghrelin have comparable binding affinities for the human ghrelin receptor (Table 2). The mean-effective concentration EC₅₀) of Compound A was 3.1 and 0.7 nM in an intra-cellular calcium mobilization assay and in an in vitro pituitary GH release assay, respectively.

In order to determine the dose response of wild type C57B mice to the ghrelin agonist Compound A, mice were orally administered (O) doses of Compound A at 1,3,10 and 20 mpk (milligrams/kilogram) and light cycye food intake was determined. When assayed for effect on food intake, Compound A significantly stimulated 4-hour food intake in ad libitum fed satiated mice during the daytime, a period of time when they normally would eat very little. The maximum stimulation on feeding was obtained by a dose of 3 mg/kg (FIG. 21).

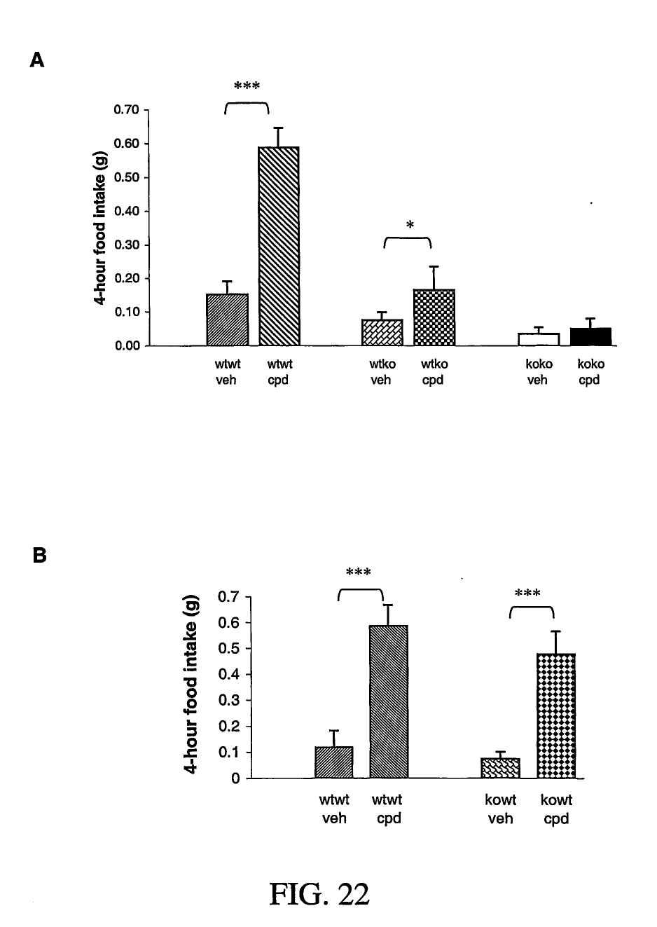

FIG. 22A shows that, when tested as a ghrelin surrogate, a single oral dose of 3 mg/kg of Compound A in 200 ml aqueous solution containing 5% Tween 80 and 0.5% methylcellulose stimulated 4-hour daytime food intake in the wild-type control mice by 4-fold as compared with saline (Compound A, 0.59±0.06 g versus vehicle, 0.15±0.04 g, P<0.001). The magnitude of stimulation was greatly diminished in the Npy^{-/-} single knockout mice (Compound A, 0.17±0.07 g versus vehicle, 0.08±0.02 g, P<0.05), and the Compound A was without effect on the Agrp^{-/-}; Npy^{-/-} double-null mice. A separate group of Agrp single knockout mice and their wild-type littermates were also tested by oral dosing of 3 mg/kg Compound A. The compound was equally efficacious in the Agrp^{-/-} single knockout mice as in the wild-type controls (FIG. 22B).

The combined data on ghrelin and Compound A indicate that removal of NPY severely compromised the feeding promotion of ghrelin, while the loss of AgRP does not by itself diminish the signaling of circulating ghrelin. The loss of NPY activity can be partially compensated by AgRP as shown by feeding stimulation in the NPY^{-/-} single knockout mice. The removal of both NPY and AgRP, however, completely abolished signaling by ghrelin or the ghrelin agonist Compound A. In summary, the feeding stimulation by peripheral ghrelin acts via NPY and AgRP, with NPY as a primary effector. This data thus clarifies that one of the in vivo function of NPY and AgRP is to relay peripheral ghrelin signaling.

1. A transgenic non-human animal whose somatic cells and germ cells are homozygous for an altered agouti-related protein (AgRP) gene which encodes a non-functional AgRP protein.

2. The transgenic animal of claim 1 wherein the animal is a mouse whose somatic cells and germ cells are homozygous for an altered agouti-related protein (AgRP) gene which encodes a non-functional AgRP protein, and further wherein said mouse exhibits reduced daytime respiratory quotient.

3. A cell line derived from a transgenic animal of claim 2.

4. A transgenic mouse whose somatic cells are heterozygous for a functional murine gene coding for an agouti-related protein (AgRP) protein and an altered AgRP gene.

5. A cell line derived from a transgenic animal according to claim 4.

6. A transgenic mouse whose somatic cells are hemizygous for an altered AgRP gene.

7. A cell line derived from a transgenic animal according to claim 6.

8. A transgenic mouse whose somatic cells and germ cells lack a functional gene coding for a murine agouti-related protein (AgRP) and which contain and express a transgene comprising a gene for a non-native AgRP protein, wherein said mouse is viable.

9. The transgenic mouse of claim 8 wherein said transgene encodes wild-type or a mutant form of human AgRP.

10. A method of producing a mouse having somatic and germ cells that lack a murine gene coding for AgRP, which comprises:
(a) providing a gene encoding an altered form of AgRP designed to target an AgRP allele of mouse embryonic stem cells;
(b) introducing the altered gene into mouse embryonic stem cells;
(c) selecting embryonic stem cells which contain the altered gene;
(d) introducing the embryonic stem cells containing the altered gene into mouse blastocysts;
(e) transplanting the injected blastocysts into a pseudopregnant mouse, and
(f) allowing the embryo to develop to term; to produce a chimeric founder transgenic mouse.

11. A transgenic non-human animal whose somatic cells and germ cells are homozygous for an altered agouti-related protein (AgRP) gene which encodes a non-functional AgRP protein and homozygous for an altered Neuropeptide Y (NPY) gene which encodes a non-functional NPY protein.

12. A cell line derived from a transgenic animal of claim 11.

13. A transgenic mouse whose somatic cells and germ cells are homozygous for an altered agouti-related protein (AgRP) gene, which encodes a non-functional AgRP protein and homozygous for an altered Neuropeptide Y (NPY) gene which encodes a non-functional NPY protein.

14. A cell line derived from a transgenic mouse of claim 13.

15. A transgenic mouse whose somatic cells are heterozygous or homozygous for an altered AgRP gene and an altered NPY gene, wherein the altered gene encodes a non-functional NPY protein.

16. A cell line derived from a transgenic animal according to claim 15.

17. A transgenic mouse whose somatic cells and germ cells lack a functional genes coding for a murine AgRP protein and a NPY protein which contains and expresses a first transgene encoding a non-native AgRP protein and a second transgene encoding a non-native NPY protein.

18. The transgenic mouse of claim 17 wherein said first transgene encodes wild-type human AgRP and said second transgene encodes wild-type human NPY.

19. A method of evaluating the effect of a candidate compound on the expenditure and/or utilization of energy, which comprises:
(a) administering the candidate compound to a transgenic mouse in accordance with claim 2; and
(b) measuring the effect of the candidate compound on the expenditure and/or utilization of energy.

20. A method of evaluating the effect of a candidate compound on the expenditure and/or utilization of energy, which comprises:
(a) administering the candidate compound to a transgenic mouse in accordance with claim 13; and
(b) measuring the effect of the candidate compound on the expenditure and/or utilization of energy.
